(19) United States
(12) Patent Application Publication
(10) Pub. No.: US 2026/0250335 A1
(43) Pub. Date: Aug. 27, 2026

(54) NCgl2747 Gene Mutant and Use Thereof in Preparation of L-lysine

(71) Applicant: NINGXIA EPPEN BIOTECH CO., LTD, Yinchuan (CN)

(72) Inventors: Gang MENG, Yinchuan (CN); Xiaoqun ZHOU, Yinchuan (CN); Aiying WEI, Yinchuan (CN); Chunguang ZHAO, Yinchuan (CN); Fengyong MA, Yinchuan (CN); Wenyou MA, Yinchuan (CN)

(73) Assignee: NINGXIA EPPEN BIOTECH CO., LTD, Yinchuan (CN)

(21) Appl. No.: 18/870,708

(22) PCT Filed: Mar. 30, 2023
(86) PCT No.: PCT/CN2023/084970

§ 371 Date: Dec. 2, 2024

(30) Foreign Application Priority Data
Jun. 1, 2022 (CN) 202210616402.9

(51) Int. Cl. C07K 14/34; C12N 15/77; C12P 13/08

(52) U.S. Cl. CPC C07K 14/34; C12N 15/77; C12P 13/08

(57) ABSTRACT

An NCg12747 gene mutant and the use thereof in the preparation of L-lysine are provided. The NCg12747 gene mutant is a DNA molecule shown in SEQ ID NO: 3, which encodes the NCg12747 mutant protein shown in SEQ ID NO: 4. Mutating the NCg12747 gene into the NCg12747 gene mutant shown in SEQ ID NO: 3 or overexpressing NCg12747 gene mutant contributes to increase in the yield and the growth rate of L-lysine. However, when the gene is weakened or knocked out, accumulation of the L-lysine is not facilitated, and the growth rate of a strain can be reduced. The NCg12747 gene mutant and the NCg12747 mutant protein encoded thereby can be used for preparing L-lysine.

### CROSS REFERENCE TO THE RELATED APPLICATIONS

This application is the national phase entry of International Application No. PCT/CN2023/084970, filed on Mar. 30, 2023, which is based upon and claims priority to Chinese Patent Application No. 202210616402.9, filed on Jun. 1, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named KJ0102S_Sequence_Listing.xml, created on Dec. 2, 2024, and is 56,632 bytes in size.

### TECHNICAL FIELD

The present application relates to an NCg12747 gene mutant and the use thereof in the preparation of L-lysine in the field of biotechnology.

### BACKGROUND

L-lysine has physiological effects such as promoting development, enhancing immunity, and improving the function of central nervous tissue. It is one of the eight basic amino acids that humans and animals cannot synthesize by themselves and are essential for growth. At present, the L-lysine is the second largest amino acid species in the world, and the main production method is fermentation. Corynebacterium glutamicum is an important lysine-producing strain. About 90% of L-lysine's industrial production is used as a nutritional supplement in the feed industry, and 10% thereof is used as a flavoring agent and a sweetener in the food industry, and as a drug intermediate in the pharmaceutical industry.

Improvements in the production of the L-lysine by the fermentation method can involve fermentation technologies such as stirring and oxygen supply; or the composition of the nutrient medium, such as sugar concentration during the fermentation; or the processing of fermentation broth into a suitable product form, e.g., by drying and pelleting the fermentation broth or ion exchange chromatography; or inherent performance properties of microorganisms themselves.

Methods used to improve the performance properties of these microorganisms include mutagenesis, and mutant selection and screening. Strains obtained in this way are resistant to antimetabolites or auxotrophic for metabolites of regulatory importance and produce the L-lysine.

### SUMMARY

The present invention aims to provide a protein that can produce L-lysine and a related biological material thereof.

In order to solve the above technical problems, the present invention first provides a protein, which is named NCg12747^{A955T}. NCg12747^{A955T} is the following A1) or A):
A1) A protein with an amino acid sequence of SEQ ID NO: 4;
A2) A fusion protein obtained by connecting a tag to an N-terminal or/and a C-terminal of A1).

In order to facilitate the purification of the protein in A1), tags as shown in the table below can be connected to an amino terminal or a carboxyl terminal of the protein consisting of the amino acid sequence shown in SEQ ID NO: 4.

TABLE
Sequence of tags

| Tags | Residue | Sequence |
|---|---|---|
| Poly-Arg | 5-6 | RRRRR |
| | (usually 5) | (SEQ ID NO: 39) |
| Poly-His | 2-10 | HHHHHH |
| | (usually 6) | (SEQ ID NO: 40) |
| FLAG | 8 | DYKDDDDK |
| | | (SEQ ID NO: 41) |
| Strep-tag II | 8 | WSHPQFEK |
| | | (SEQ ID NO: 42) |
| c-myc | 10 | EQKLISEEDL |
| | | (SEQ ID NO: 43) |

The present invention further provides a biological material related to NCg12747^{A955T} which is any one of the following B1) to B4):
B1) A nucleic acid molecule encoded as NCg12747^{A955T};
B2) An expression cassette containing the nucleic acid molecule in B1);
B3) A recombinant vector containing the nucleic acid molecule in B1), or a recombinant vector containing the expression cassette in B2);
B4) A recombinant microorganism containing the nucleic acid molecule in B1), or a recombinant microorganism containing the expression cassette in B2), or a recombinant microorganism containing the recombinant vector in B3).

In the above biological materials, the nucleic acid molecule in B1) may be the following b11) or b12) or b13):
b11) A DNA molecule shown in SEQ ID NO: 3 in a sequence listing;
b12) A DNA molecule that has 75% or more identity with a nucleotide sequence defined by b11) and is encoded as NCg12747^{A955T};
b13) A genomic DNA molecule that hybridizes with the nucleotide sequence defined by b11) or b12) under stringent conditions and is encoded as NCg12747^{A955T}.

Wherein the nucleic acid molecule may be DNA, such as cDNA, genomic DNA or recombinant DNA; the nucleic acid molecule may also be RNA, such as mRNA or hnRNA.

Those ordinary skilled in the art can easily use known methods, such as directed evolution and point mutation, to mutate the nucleotide sequence of the present invention which encodes the NCg12747^{A955T} protein. Those nucleotides that have been artificially modified and have 75% or higher identity with the nucleotide sequence of the NCg12747^{A955T} protein of the present invention, as long as they encode the NCg12747^{A955T} protein and have the function of the NCg12747^{A955T} protein, are all derived from the nucleotide sequences of the present invention and are equivalent to the sequences of the present invention.

The term “identity” used herein refers to sequence similarity to a native nucleic acid sequence. The “identity” includes nucleotide sequences having 75% or higher, or 85% or higher, or 90% or higher, or 95% or greater identity with the nucleotide sequence which encodes the protein consisting of the amino acid sequence shown in SEQ ID NO: 4 in the present invention. The identity may be evaluated with the naked eye or using computer software. Using the computer software, the identity between two or more sequences may be expressed as a percentage (%), which may be used to evaluate the identity between related sequences.

In the above biological materials, the stringent conditions can be as follows: hybridizing in a mixed solution of 7% sodium dodecyl sulfate (SDS), 0.5M NaPO₄ and 1 mM EDTA at 50° C., and rinsing in 50° C., 2×SSC, 0.1% SDS; or hybridizing in a mixed solution of 7% SDS, 0.5M NaPO₄ and ImM EDTA at 50° C., and rinsing in 50° C., 1×SSC, 0.1% SDS; or hybridizing in a mixed solution of 7% SDS, 0.5M NaPO₄ and ImM EDTA at 50° C., and rinsing in 50° C., 0.5×SSC, 0.1% SDS; or hybridizing in a mixed solution of 7% SDS, 0.5M NaPO₄ and 1 mM EDTA at 50° C., and rinsing in 50° C., 0.1×SSC, 0.1% SDS; or hybridizing in a mixed solution of 7% SDS, 0.5M NaPO₄ and 1 mM EDTA at 50° C., and rinsing in 65° C., 0.1×SSC, 0.1% SDS; or hybridizing in 6×SSC, 0.5% SDS solution at 65° C., and then washing membrane once with 2×SSC, 0.1% SDS and 1×SSC, 0.1% SDS respectively; or hybridizing and washing the membrane twice in 2×SSC, 0.1% SDS solution at 68° C., 5 min each time, and then hybridizing and washing the membrane twice in 0.5×SSC, 0.1% SDS solution at 68° C., 15 min each time; or hybridizing and washing the membrane in 0.1×SSPE (or 0.1×SSC), 0.1% SDS solution at 65° C.

The above 75% or above identity may be 80%, 85%, 90% or 95% or above identity.

In the above biological materials, the expression cassette (NCg12747^{A955T} gene expression cassette) containing the nucleic acid molecule encoding the NCg12747^{A955T} protein in B2) refers to DNA that can express the NCg12747^{A955T} protein in a host cell. The DNA can not only include a promoter that promotes the transcription of the NCg12747^{A955T} gene, but also include a terminator that terminates the transcription of the NCg12747^{A955T} gene. Furthermore, the expression cassette may also include an enhancer sequence.

In the above biological materials, the expression cassette in B2) may specifically be the DNA molecule shown in SEQ ID NO: 8.

Existing expression vectors can be used to construct a recombinant vector containing the NCg12747^{A955T} gene expression cassette.

In the above biological materials, the vector may be a plasmid, cosmid, phage or a viral vector. The plasmid may specifically be a pK18mobsacB vector or a pXMJ19 vector.

The recombinant vector in B3) may be a recombinant vector pK18-NCg12747A^{955T}, pK18-NCg12747^{A955T}OE or pXMJ19-NCg12747^{A955T}

The recombinant vector pK18-NCg12747^{A955T} is a recombinant vector obtained by replacing the fragment (small fragment) between Xbal I and BamH I recognition sites of the pK18mobsacB vector with the DNA fragment shown in SEQ ID NO: 5 in the sequence listing, while keeping other sequences of the pK18mobsacB vector unchanged. The recombinant vector pK18-NCg12747^{A955T} contains the mutation sites (A-T) of the mutant gene NCg12747^{A955T} shown in SEQ ID NO: 3.

The pK18-NCg12747^{A955T}OE is a recombinant vector obtained by inserting the expression cassette in B2) between recognition sequences of Xbal I and BamH I of pK18mobsacB.

The pXMJ19-NCg12747^{A955T} is a recombinant vector obtained by inserting the expression cassette in B2) into the pXMJ19 vector.

In the above biological materials, the microorganism may be yeast, bacteria, algae or fungi. Wherein, the bacteria may be Corynebacterium glutamicum, Escherichia coli, Pantoea ananatis, Bacillus brevis or Brevis lactobacillus.

In one embodiment of the present invention, the Corynebacterium glutamicum is Corynebacterium glutamicum YP097158 or Corynebacterium glutamicum ATCC13032.

The recombinant microorganism in B4) is a recombinant microorganism obtained by replacing the NCg12747 gene in the microorganism containing the NCg12747 gene shown in SEQ ID NO: 1 with the nucleic acid molecule in B1), or introducing the nucleic acid molecule in B1) into the microorganism and expressing it.

In embodiments of the present invention, the recombinant microorganism is a recombinant strain YPL-NCg12747-1, a recombinant strain L2747-1, a recombinant strain YPL-NCg12747-3, a recombinant strain L2747-3, a recombinant strain YPL-NCg12747-5 or a recombinant strain L2747-5.

The recombinant strain YPL-NCg12747-1 is a strain obtained by replacing the NCg12747 gene of Corynebacterium glutamicum YP097158 with the NCg12747^{A955T} gene while keeping other sequences unchanged.

The recombinant strain L2747-1 is a strain obtained by replacing the NCgl2747 gene of ATCC13032 with the NCg12747^{A955T} gene while keeping other sequences unchanged.

The recombinant strain YPL-NCg12747-3 is a recombinant strain obtained by replacing a spacer region of an upstream homologous arm NCgl1741 and a downstream homologous arm NCg11742 in a genome of the Corynebacterium glutamicum YP097158 with the NCg12747^{A955T} gene and a promoter thereof (i.e., positions 1-331 in SEQ ID NO: 8 in the sequence listing), while keeping other nucleotides in the genome of the Corynebacterium glutamicum YP097158 unchanged.

The recombinant strain L2747-3 is a recombinant strain obtained by replacing the spacer region of the upstream homologous arm NCgl1741 and the downstream homologous arm NCg11742 in a genome of the Corynebacterium glutamicum ATCC13032 with the NCg12747^{A955T} gene and a promoter thereof (i.e., positions 1-331 in SEQ ID NO: 8 in the sequence listing), while keeping other nucleotides in the genome of the Corynebacterium glutamicum ATCC13032 unchanged.

The recombinant strain YPL-NCg12747-5 is a recombinant strain obtained by introducing the pXMJ19-NCg12747^{A955T} into the Corynebacterium glutamicum YP097158.

The recombinant strain L2747-5 is a recombinant strain obtained by introducing the pXMJ19-NCg12747^{A955T} into the Corynebacterium glutamicum ATCC13032.

The present invention further provides a method for preparing L-lysine, which includes: expressing NCg12747^{A955T} in a recipient biological cell, or increasing the content or activity of the NCg12747^{A955T} in the recipient biological cell, or increasing the content or activity of the protein shown in SEQ ID NO: 2 in the recipient biological cell to obtain a recombinant biological cell; and culturing the recombinant biological cell to obtain the L-lysine.

In the above method, the biological cell may be yeast, bacterium, algae, fungi, a plant cell or an animal cell capable of synthesizing the L-lysine.

The bacterium is Corynebacterium glutamicum, such as Corynebacterium glutamicum YP097158.

The bacterium in the present invention includes but not limited to the Corynebacterium glutamicum. Any containing the Nog12747 gene shown in SEQ ID NO: 1 in the sequence listing and can synthesize the L-lysine can produce the L-lysine using the NCg12747 mutant protein shown in SEQ ID NO: 1 and related biological materials thereof in the present invention. For example, the bacterium may be Corynebacterium glutamicum, Escherichia coli, Pantoea ananatis, Bacillus brevis or Brevis lactobacillus.

The above method can be realized by introducing an encoding gene of the NCg12747^{A955T} into the recipient biological cell and expressing it, or introducing an encoding gene of the protein shown in SEQ ID NO: 2 into the recipient biological cell and expressing it;
Or, the recipient biological cell contains a DNA molecule shown in SEQ ID NO: 1, and the method is realized by replacing the DNA molecule shown in SEQ ID NO: 1 in the recipient biological cell with a DNA molecule shown in SEQ ID NO: 3.

In the above method, the recombinant biological cell may be cultured using a culture medium capable of growing the recombinant biological cell;
And/or, the recombinant biological cell is cultured using conditions that enable the growth of the recombinant biological cell.

The recombinant biological cell may be the recombinant microorganism as described above.

The present invention further provides a product for preparing L-lysine, which contains (or of which the active ingredient is) NCg12747^{A955T} or the biological material.

NCg12747^{A955T} or the biological material of the present invention may be used to produce a variety of products, including but not limited to lysine in the embodiments. The amino acids produced may also be glutamic acid, valine, glycine, alanine, leucine, isoleucine, methionine, proline, tryptophan, serine, tyrosine, cysteine, phenylalanine, asparagine, glutamine, threonine, aspartic acid, arginine and histidine, shikimic acid, protocatechuic acid, succinic acid, a-Ketoglutaric acid, citric acid, ornithine, and citrulline. When producing various target products, the production of the target products can be achieved by placing the NCg12747^{A955T} of the present invention in a synthesis pathway of the target products.

### Instructions for Preservation of Biological Materials

Classification and naming: Corynebacterium glutamicum
Strain number: YP097158
Name of depository authority: China General Microbiological Culture Collection Center
Abbreviation: CGMCC
Address: No. 3, No. 1, Beichen West Road, Chaoyang District, Beijing 100101, China
Collection date: Aug. 16, 2016
Collection center registration number: CGMCC No. 12856

### DETAILED DESCRIPTION OF THE EMBODIMENTS

The present invention will be further described in detail below with reference to specific embodiments. The embodiments given are only for illustrating the present invention and are not intended to limit the scope of the present invention. The embodiments provided below can serve as a guide for those ordinary skilled in the art to make further improvements, and do not limit the present invention in any way.

The experimental methods in the following embodiments are all conventional methods unless otherwise specified. The materials, reagents, instruments, etc. used in the following embodiments can be obtained from commercial sources unless otherwise specified. The quantitative experiments in the following embodiments are repeated three times, and the results are averaged. In the following embodiments, unless otherwise specified, the first position of each nucleotide sequence in the sequence listing is the 5′ terminal nucleotide of the corresponding DNA/RNA, and the last position thereof is the 3′ terminal nucleotide of the corresponding DNA/RNA.

### Embodiment 1. Construction of a Recombinant Vector Containing an NCg12747 Gene Coding Region with Point Mutation

Based on the genome sequence of Corynebacterium glutamicum ATCC13032 published by NCBI, two pairs of primers for amplifying the NCg12747 gene coding region were designed and synthesized. The point mutation was introduced into the NCg12747 gene coding region (SEQ ID NO: 1) of the Corynebacterium glutamicum YP097158 (collection number: CGMCC No. 12856, collection date: Aug. 16, 2016, depository authority: Institute of Microbiology, Chinese Academy of Sciences, No. 3, No. 1, Beichen West Road, Chaoyang District, Beijing, tel.: 010-64807355) and a wild type Corynebacterium glutamicum strain ATCC13032. The point mutation refers to mutating the adenine (A) at position 955 in the nucleotide sequence (SEQ ID NO: 1) of the NCg12747 gene into thymine (T) to obtain the DNA molecule shown in SEQ ID NO: 3 (the mutated NCgl2747 gone, recorded as an NCg12747^{A955T} gene).

Wherein the amino acid sequence which is encoded by the NCg12747 gene shown in SEQ ID NO: 1 is SEQ ID NO: 2.

The amino acid sequence which is encoded by the NCg12747^{A955T} gene shown in SEQ ID NO: 3 is a mutant protein (i.e., NCgl2747^{A955T} protein) in SEQ ID NO: 4. The phenylalanine (F) at position 319 in the amino acid sequence (SEQ ID NO: 4) of the NCg12747^{I319F} protein is mutated from the isoleucine (I) at position 319 of the NCg12747 protein.

The recombinant vector is constructed using an NEBuilder assembly technology. The primers are designed as follows (synthesized by Shanghai Invitrogen Company). The nucleotides in bold font are the mutation positions:

P1:
(SEQ ID NO: 15)
5′-CAGTGCCAAGCTTGCATGCCTGCAGGTCGACTCTAGTCACCCGCTACGAAGTTGT-3′
(the underlined nucleotide sequence is the sequence on pK18),
P2:
(SEQ ID NO: 16)
5′-CTACCTGAAAAGCGTGCTGAGCTGGGACATTTG-3′,
P3:
(SEQ ID NO: 17)
5′-CAAATGTCCCAGCTCAGCACGCTTTTCAGGTAGCTCTCGGTG-3′,
P4:
(SEQ ID NO: 18)
5′-CAGCTATGACCATGATTACGAATTCGAGCTCGGTACCCTTGTTCTACGAATGCCCAC-3′
(the underlined nucleotide sequence is the sequence on pK18).

Construction method: Using Corynebacterium glutamicum ATCC13032 as a template, PCR amplification was performed with primers P1 and P2, P3 and P4 respectively, and two DNA fragments (NCg12747 Up and NCg12747 Down) of the NCg12747 gene coding region, which have mutant nucleotides and sizes of 624 bp and 705 bp respectively, were obtained.

The above two DNA fragments (NCgl2747 Up and NCgl2747 Down) were separated and purified by agarose gel electrophoresis, and then ligated with the purified pK18mobsacB plasmid (Addgene Company) subject to enzyme digestion (Xbal I and BamH I) using NEBuilder enzyme (NEB Company) at 50° C. for 30 min. The single clone grown after the ligated product was transformed into Escherichia coli DHSa was PCR-identified using primers M13F/M13R(M13F: 5′-TGTAAAACGACGGCCAGT-3′ (SEQ ID NO: 19), M13R: 5′-CAGGAAACAGCTATGACC-3′ (SEQ ID NO: 20)), the plasmid was extracted to obtain a positive recombinant vector with correct sequence, which was recorded as pK18-NCg12747^{A955T} The recombinant vector contains a kanamycin resistance marker.

The NCg12747^{A955T} Up-Down DNA in the recombinant vector pK18-NCg12747^{A955T} has a size of 1296 bp (SEQ ID NO: 5) and contains mutation sites (A-T), which would lead to the mutation of the adenine (A) at position 955 in the NCg12747 gene coding region in Corynebacterium glutamicum YP097158 into thymine (T), ultimately resulting in the change of isoleucine (I) of the encoded protein at position 319 to phenylalanine (F).

The recombinant vector pK18-NCg12747^{A955T} is a recombinant vector obtained by replacing the fragment (small fragment) between Xbal I and BamH I recognition sites of the pK18mobsacB vector with the DNA fragment shown in SEQ ID NO: 5 in the sequence listing, while keeping other sequences of the pK18mobsacB vector unchanged. The recombinant vector pK18-NCg12747^{A955T} contains the mutation sites (A-T) of the mutant gene NCg12747^{A955T} shown in SEQ ID NO: 3.

### Embodiment 2. Construction of an Engineering Strain Containing a Gene NCg12747^{A955T}

The allelic substitution plasmid (pK18-NCg12747^{A955T}) in Embodiment 1 was transformed into L-lysine-producing strain Corynebacterium glutamicum YP097158 by electric shock (for its construction method, please refer to WO2014121669A1; upon confirmation by sequencing, a wild type NCgl2747 gene coding region was retained on the chromosome of this strain) and the wild type Corynebacterium glutamicum strain ATCC13032, and cultured on a solid culture plate containing kanamycin (refer to Table 1 for the composition and culture conditions of culture medium). The single colonies produced by culturing were identified by a primer P1 and a universal primer M13R in Embodiment 1 respectively, and the strain that could amplify a 1384 bp band was a positive strain. The positive strain was cultured on a culture medium containing 15% sucrose (the culture medium was obtained by increasing the concentration of sucrose in the culture medium in Table 1 to 15 g/L), the single colonies produced by culturing were cultured on a culture medium containing and not containing kanamycin respectively, and grew on the culture medium not containing kanamycin, while the strains that did not grow on the culture medium containing kanamycin was further PCR-amplified using the following primers (synthesized by Shanghai Invitrogen Company):

P5:
(SEQ ID NO: 21)
5′-TCTATCCAAGGCATACCGC-3′;
P6:
(SEQ ID NO: 22)
5′-TCCCATTGGTTTCACACAG-3′.

The obtained DNA fragment (280 bp) was processed (95° C. high temperature denaturation for 10 min and rapid ice bath for 5 min) and then subject to Single-Strand Conformation Polymorphis (SSCP) electrophoresis (using the amplified fragment of plasmid pK18-NCg12747^{A1089C} as a positive control, Corynebacterium glutamicum ATCC13032 amplified fragment as a negative control, and water as a blank control). Please refer to Table 2 for the preparation and electrophoresis conditions of PAGE for SSCP electrophoresis. Due to different fragment structures and different electrophoresis positions, strains whose electrophoresis positions are inconsistent with the position of the negative control fragment and consistent with the position of the positive control fragment are strains with successful allelic substitution. The NCg12747^{A955T} gene fragment of the positive strain was again PCR-amplified by primers P5/P6, and ligated to a PMD19-T vector for sequencing. Through sequence comparison, the strain with mutation (A-T) in a base sequence was a positive strain with successful allelic substitution. The positive strains obtained from Corynebacterium glutamicum YP097158 and wild type Corynebacterium glutamicum strain ATCC13032 were named YPL-NCg12747-1 and L2747-1 respectively.

The recombinant strains YPL-NCg12747-1 and L2747-1 contain the mutated gene NCg12747^{A955T} shown in SEQ ID NO: 3, and can express the protein shown in SEQ ID NO: 4. The only difference between the recombinant strain YPL-NCg12747-1 and the Corynebacterium glutamicum YP097158 is that: YPL-NCg12747-1 replaces the NCg12747 gene of the Corynebacterium glutamicum YP097158 with the NCg12747^{A955T} gene while keeping the strains obtained with other sequences unchanged. The only difference between the recombinant strain L2747-1 and ATCC13032 is that: L2747-1 is a strain obtained by replacing the NCg12747 gene of ATCC13032 with the NCg12747^{A955T} gene while keeping other sequences unchanged.

TABLE 1
Composition and culture conditions of culture medium

| | Composition | Content | |
|---|---|---|---|
| Culture medium | Sucrose | 10 | g/L |
| | Polypeptone | 10 | g/L |
| | Beef extract | 10 | g/L |
| | Yeast powder | 5 | g/L |
| | Carbamide | 2 | g/L |
| | Sodium chloride | 2.5 | g/L |
| | Agar powder | 20 | g/L |

| | |
|---|---|
| | Water |
| | pH 7.0 |
| Culture condition | Culture temperature: 32° C. |
| | Culture time: 40 h |

TABLE 2
Preparation and electrophoresis conditions
of PAGE for SSCP electrophoresis

| | | Usage (the concentration | |
|---|---|---|---|
| | Composition | of the acrylamide is 8%) | |
| PAGE | 40% acrylamide | 8 | mL |
| | ddH₂O | 26 | mL |
| | Glycerin | 4 | mL |
| | 10 × TBE | 2 | mL |
| | TEMED | 40 | μL |
| | 10% APS | 600 | μL |

| | |
|---|---|
| Electrophoresis | Placing an electrophoresis tank in ice, and |
| condition | using 1 × TBE buffer solution, 120 V |
| | voltage, and an electrophresis time of 10 h |

### Embodiment 3. Construction of an Engineering Strain Overexpressing an NCg12747 Gene or an NCgl2747^{A955T} Gene on a Genome

Based on the genome sequence of Corynebacterium glutamicum ATCC13032 published by NCBI, three pairs of primers for amplifying the fragments of the upstream and downstream homologous arms and the NCg12747 or an NCg12747^{A955T} gene coding region and a promoter region were designed and synthesized. The NCg12747 or NCg12747^{A955T} gene was inserted into the Corynebacterium glutamicum YP097158 and the wild type Corynebacterium glutamicum ATCC13032.

The primers are designed as follows (synthesized by Shanghai Invitrogen Company):

P7:
(SEQ ID NO: 23)
5′-CAGTGCCAAGCTTGCATGCCTGCAGGTCGACTCTAGAATGCGTTCTGGACTGAGG-3′
(the underlined nucleotide sequence is the sequence on pK18),
P8:
(SEQ ID NO: 24)
5′-GATTGAGTAGCAAGTGCTTTGGTGCACCGAGAACAGATG-3′,
P9:
(SEQ ID NO: 25)
5′-CATCTGTTCTCGGTGCACCAAAGCACTTGCTACTCAATC-3′,
P10:
(SEQ ID NO: 26)
5′-GATTTAATTGCGCCATCTGCTACTGCTTGTAAGTGGACAGG-3′,
P11:
(SEQ ID NO: 27)
5′-CCTGTCCACTTACAAGCAGTAGCAGATGGCGCAATTAAATC-3′,
P12:
(SEQ ID NO: 28)
5′-CAGCTATGACCATGATTACGAATTCGAGCTCGGTACCCGCTATGACACCTTCAACGGATC-3
(the underlined nucleotide sequence is the sequence on pK18).

Construction method: Using the Corynebacterium glutamicum ATCC13032 as a template, PCR amplification was performed with primers P7/P8, P9/P10, and P11/P12 to obtain an upstream homologous arm fragment 763 bp (corresponding to a partial coding region of an NCg11740 gene of the Corynebacterium glutamicum ATCC13032 and an NCgl1741 gene and a promoter region thereof, with the sequence shown in SEQ ID NO: 6), an NCg12747 gene and a promoter fragment 1645 bp thereof (the sequence is shown in SEQ ID NO: 7), and a downstream homologous arm fragment 596 bp (corresponding to a partial coding region of an NCgl1742 gene of the Corynebacterium glutamicum ATCC13032, with the sequence shown in SEQ ID NO: 9). After the PCR reaction, the three fragments amplified from each template were separately recovered by electrophoresis using a column type DNA gel recovery kit. The three recovered fragments were ligated with the purified pK18mobsacB plasmid (Addgene Company) subject to Xbal I and BamH I enzyme digestion using NEBuilder enzyme (NEB Company) at 50° C. for 30 min. The single clone grown after the transformation of the ligated product was PCR-identified using an M13 primer (M13F: 5′-TGTAAAACGGACGGCCAGT-3′ (SEQ ID NO: 19), M13R: 5′-CAGGAAACAGCTATGACC-3′ (SEQ ID NO: 20)) to obtain a positive integration plasmid (recombinant vector). The resulting recombinant vector was pK18-NCg12747OE, the positive integration plasmid contains a kanamycin resistance marker, and the plasmid integrated into the recombinant on the genome can be obtained through kanamycin screening.

In SEQ ID NO: 7, the promoters of the NCgl1741 gene are located at positions 1-331, and the NCgl1741 gene is located at positions 332-1645.

Using the Corynebacterium glutamicum YPL-NCg12747-1 as a template, PCR amplification was performed with primers P7/P8, P9/P10, and P11/P12 to obtain an upstream homologous arm fragment 763 bp (corresponding to a partial coding region of an NCgl1740 gene of the Corynebacterium glutamicum ATCC13032 and an NCgl1741 gene and a promoter region thereof, with the sequence shown in SEQ ID NO: 6), an NCg12747^{A955T} gene and a promoter fragment 1645 bp thereof (the sequence is shown in SEQ ID NO: 8), and a downstream homologous arm fragment 596 bp (corresponding to a partial coding region of an NCgl1742 gene of the Corynebacterium glutamicum ATCC13032, with the sequence shown in SEQ ID NO: 9). After the PCR reaction, the three fragments amplified from each template were separately recovered by electrophoresis using a column type DNA gel recovery kit. The three recovered fragments were ligated with the purified pK18mobsacB plasmid (Addgene Company) subject to Xbal I and BamH I enzyme digestion using NEBuilder enzyme (NEB Company) at 50° C. for 30 min. The single clone grown after the transformation of the ligated product was PCR-identified using an M13 primer (M13F: 5′-TGTAAAACGACGGCCAGT-3′ (SEQ ID NO: 19), M13R: 5′-CAGGAAACAGCTATGACC-3′ (SEQ ID NO: 20)) to obtain a positive integration plasmid (recombinant vector). The resulting recombinant vector was pK18-NCg12747^{A955T}OE, the positive integration plasmid contains a kanamycin resistance marker, and the plasmid integrated into the recombinant on the genome can be obtained through kanamycin screening.

In SEQ ID NO: 8, the promoters of the NCg12747^{A955T} gene are located at positions 1-331, and the NCg12747^{A955T} gene is located at positions 332-1645.

The correctly sequenced integration plasmids (pK18-NCg12747OE, pK18-NCg12747^{A955T}OE) were electrotransformed into Corynebacterium glutamicum strain YP097158 and wild type Corynebacterium glutamicum ATCC13032 respectively, and cultured in the culture medium. Please refer to Table 1 for the composition and culture conditions of the culture medium. The single colonies produced by culturing were PCR-identified using primers P13/P14. The PCR-amplified strains containing a fragment with a size of 1959 bp (the sequence is shown in SEQ ID NO: 10) were positive strains, and those that failed to amplify the fragment were original strains. The positive strains were streaked and cultured on a solid culture plate containing 15% sucrose (the culture medium was obtained by increasing the concentration of sucrose in the culture medium in Table 1 to 15 g/L), and the single colonies produced by culturing were further PCR-identified using primers P15/P16, and the amplified strains with a size of 1600 bp (the sequence is shown in SEQ ID NO: 11) was positive strains that NCg12747 or NCg12747^{A1089C} gene and a promoter thereof were integrated into a spacer region of an upstream homologous arm NCgl1741 and a downstream homologous arm Nogl1742 in a genome of the Corynebacterium glutamicum. Strains obtained by taking Corynebacterium glutamicum YP097158 as an original strain were named YPL-NCg12747-2 (not containing mutation sites) and YPL-NCg12747-3 (containing mutation sites) respectively, and strains obtained by taking Corynebacterium glutamicum ATCC13032 as an original stain were named L2747-2 (not containing mutation sites) and L2747-3 (containing mutation sites) respectively.

The recombinant strain YPL-NCg12747-2 contains double copies of the NCgl2747 gene shown in SEQ ID NO: 1. Specifically, the recombinant strain YPL-NCg12747-2 is a recombinant strain by replacing the spacer region of the upstream homologous arm NCgl1741 and the downstream homologous arm NCg11742 in the genome of the Corynebacterium glutamicum YP097158 with the NCg12747 gene and the promoter thereof (i.e., positions 1-331 in SEQ ID NO: 7 in the sequence listing), while keeping other nucleotides in the genome of the Corynebacterium glutamicum YP097158 unchanged. The recombinant bacteria containing double copies of the NCg12747 gene can significantly and stably increase the expression quantity of the NCg12747 gene.

The recombinant strain L2747-2 contains double copies of the NCg12747 gene shown in SEQ ID NO: 1. Specifically, the recombinant strain L2747-2 is a recombinant strain by replacing the spacer region of the upstream homologous arm NCgl1741 and the downstream homologous arm NCgl1742 in the genome of the Corynebacterium glutamicum ATCC13032 with the NCg12747 gene and the promoter thereof (i.e., positions 1-331 in SEQ ID NO: 7 in the sequence listing), while keeping other nucleotides in the genome of the Corynebacterium glutamicum ATCC13032 unchanged. The recombinant bacteria containing double copies of the NCg12747 gene can significantly and stably increase the expression quantity of the NCg12747 gene.

The recombinant strain YPL-NCg12747-3 contains the mutated NCg12747^{A955T} gene shown in SEQ ID NO: 3. Specifically, the recombinant strain YPL-NCg12747-3 is a recombinant strain by replacing the spacer region of the upstream homologous arm NCgl1741 and the downstream homologous arm NCgl1742 in the genome of the Corynebacterium glutamicum YP097158 with the NCg12747^{A955T} gene and the promoter thereof (i.e., positions 1-331 in SEQ ID NO: 8 in the sequence listing), while keeping other nucleotides in the genome of the Corynebacterium glutamicum YP097158 unchanged.

The recombinant strain L2747-3 contains the mutated NCg12747^{A955T} gene shown in SEQ ID NO: 3. Specifically, the recombinant strain L2747-3 is a recombinant strain by replacing the spacer region of the upstream homologous arm NCgl1741 and the downstream homologous arm NCg11742 in the genome of the Corynebacterium glutamicum ATCC13032 with the NCg12747^{A955T} gene and the promoter thereof (i.e., positions 1-331 in SEQ ID NO: 8 in the sequence listing), while keeping other nucleotides in the genome of the Corynebacterium glutamicum ATCC13032 unchanged.

The PCR-identified primers are as follows:
P13:
(SEQ ID NO: 29)
5′-TCCAAGGAAGATACACGCC-3′ (corresponding to the outside of the upstream
homologous arm NCgl1740),
P14:
(SEQ ID NO: 30)
5′-GGTCGTAGATTTCATCGGC-3′ (corresponding to the inside of the Ncgl2747
gene),
P15:
(SEQ ID NO: 31)
5′-CAACGGTGTCTCAGAACTAATC-3′ (corresponding to the inside of the
Ncgl2747 gene),
P16:
(SEQ ID NO: 32)
5′-TGGTCGTTGGAATCTTGC-3′ (corresponding to the outside of the downstream
homologous arm NCgl1742).

### Embodiment 4. Construction of an engineering strain overexpressing an NCg12747 gene or an NCg12747^{A955T} gene on a plasmid

Based on the genome sequence of Corynebacterium glutamicum ATCC13032 published by NCBI, a pair of primers for amplifying the NCg12747 or NCg12747^{A1089C} gene coding region and a promoter region were designed and synthesized. The primers are designed as follows (synthesized by Shanghai Invitrogen Company):

P17:
(SEQ ID NO: 33)
5′-GCTTGCATGCCTGCAGGTCGACTCTAGAGGATCCCCCAAAGCACTTGCTACTCAATC-3′
(the underlined nucleotide sequence is the sequence on pXMJ19),
P18:
(SEQ ID NO: 34)
5′-ATCAGGCTGAAAATCTTCTCTCATCCGCCAAAACCTACTGCTTGTAAGTGGACAGG-3′
(the underlined nucleotide sequence is the sequence on pXMJ19).

Construction method: Using Corynebacterium glutamicum ATCC13032 and YPL-NCg12747-1 as templates respectively, PCR amplification was performed with primers P17/P18 to obtain the NCg12747 gene and a promoter fragment thereof (the sequence is shown in SEQ ID NO: 12) and the NCg12747^{A955T} gene and a promoter fragment thereof 1715 bp (the sequence is shown in SEQ ID NO: 13). The amplified product was electrophoresed and purified and recovered using a column type DNA gel recovery kit. The recovered DNA fragments were ligated with a shuttle plasmid pXMJ19 recovered through EcoR I enzyme digestion using NEBuilder enzyme (NEB Company) at 50° C. for 30 min. The single clone grown after the transformation of the ligated product was PCR-identified using a primer M13 to obtain the positive overexpression plasmid pXMJ19-NCg12747 (containing the NCgl2747 gene) and pXMJ19-NCg12747^{A955T} (containing the NCg12747^{A955T} gene), and the positive plasmid was sent for sequencing. Because the plasmid contains a chloramphenicol resistance marker, chloramphenicol can be used to screen whether the plasmid has been transformed into the strain.

In SEQ ID NO: 12, the promoters of the NCg12747 gene are located at positions 37-367, and the NCg12747 gene is located at positions 368-1681.

In SEQ ID NO: 13, the promoters of the NCg12747^{A955T} gene are located at positions 37-367, and the NCg12747^{A955T} gene is located at positions 368-1681.

The correctly sequenced pXMJ19-NCg12747 and pXMJ19-NCg12747^{A955T} plasmids were electrotransformed into Corynebacterium glutamicum YP097158 and wild type Corynebacterium glutamicum ATCC13032, respectively, and cultured in the culture medium. Please refer to Table 1 for the composition and culture conditions of the culture medium. The single colonies produced by culturing were PCR-identified using primers M13R(−48)/P18 (M13R(−48): AGCGGATAACAATTTCACACAGGA). The strains obtained by taking the Corynebacterium glutamicum YP097158 as an original strain were named YPL-NCg12747-4 (containing plasmid pXMJ19-NCg12747) and YPL-NCg12747-5 (containing plasmid pXMJ19-NCg12747^{A955T}) respectively. The strains obtained by taking the Corynebacterium glutamicum ATCC13032 as the initial strains were named L2747-4 (containing plasmid pXMJ19-NCg12747) and L2747-5 (containing plasmid pXMJ19-NCg12747^{A955T}) respectively.

The recombinant strains YPL-NCg12747-4 and L2747-4 contain the plasmid carrying the NCg12747 gene shown in SEQ ID NO: 1;

The recombinant strains YPL-NCg12747-5 and L2747-5 contain the plasmid carrying the mutated NCg12747^{A955T} gene shown in SEQ ID NO: 3.

### Embodiment 5. Construction of an Engineering Strain Lacking an NCg12747 Gene on a Genome

Based on the genome sequence of Corynebacterium glutamicum ATCC13032 published by NCBI, two pairs of primers for amplifying the fragments at both ends of the NCg12747 gene coding region were synthesized and used as fragments of the upstream and downstream homologous arms. The primers are designed as follows (synthesized by Shanghai Invitrogen Company):

P19:
(SEQ ID NO: 35)
5′-CAGTGCCAAGCTTGCATGCCTGCAGGTCGACTCTAGGCAGCAAGTAGCCTGGGTAT-3′
(the underlined nucleotide sequence is the sequence on pK18),
P20:
(SEQ ID NO: 36)
5′-AGAAGATGAAGGACGTGGGTAACTTCCTGTCCACT-3′,
P21:
(SEQ ID NO: 37)
5′-AGTGGACAGGAAGTTACCCACGTCCTTCATCTTCTCCGAC-3′,
P22:
(SEQ ID NO: 38)
5′-CAGCTATGACCATGATTACGAATTCGAGCTCGGTACCCACGCAACACTTGATGGAGT-3′
(the underlined nucleotide sequence is the sequence on pK18).

Construction method: Using Corynebacterium glutamicum ATCC13032 as a template, PCR amplification was performed with primers P19/P20 and P21/P22 respectively, to obtain an upstream homologous arm fragment 647 bp and a downstream homologous arm fragment 753 bp of the knockout Ncg12747. The amplified product was electrophoresed and purified using a column DNA gel recovery kit. The recovered DNA fragment was ligated with the purified pK18mobsacB plasmid (Addgene Company) subject to Xbal I/BamH I enzyme digestion using NEBuilder enzyme (NEB Company) at 50° C. for 30 min. The single clone grown after the transformation of the ligated product was PCR-identified using a primer M13 to obtain the positive knockout vector pK18-ΔNCg12747. The plasmid contains the homologous arm fragment 1400 bp of the entire knockout Ncg12747 (the sequence is shown in SEQ ID NO: 14) and kanamycin resistance as a selection marker, and the plasmid was sent for sequencing.

The correctly sequenced knockout plasmid pK18-ΔNCg12747 was electrotransformed into Corynebacterium glutamicum YP097158 and wild type Corynebacterium glutamicum ATCC13032, and cultured in the culture medium. Please refer to Table 1 for the composition and culture conditions of the culture medium. The single colonies produced by culturing were PCR-identified using primers P19/P22: strains that simultaneously amplified 1400 bp and 2549 bp bands were positive strains, and strains that only amplified 2549 bp bands were original strains. The positive strains were screened on a 15% sucrose solid medium and then cultured on a culture medium containing kanamycin and not containing kanamycin respectively. The strains were selected to grow on the culture medium not containing kanamycin, while the strains that did not grow on the culture medium containing kanamycin were further PCR-identified using primers P19/P22, and the strains that amplified the 1400 bp bands were positive strains with the NCg12747 gene coding region knocked out. The NCg12747 fragment of the positive strain was again PCR-amplified using the primers P19/P22 and ligated to the pMD19-T vector for sequencing. The correctly sequenced strains were named YPL-NCg12747-6 (the NCg12747 gene on the genome of the Corynebacterium glutamicum YP097158 was knocked out) and L2747-6 (the NCg12747 gene on the genome of the wild type Corynebacterium glutamicum ATCC13032 was knocked out).

### Embodiment 6. L-Lysine Fermentation Experiment

The strains constructed in Embodiments 1-5 and the original strains of the Corynebacterium glutamicum YP097158 and ATCC13032 were subject to a fermentation experiment in a culture medium shown in Table 3 with a control process shown in Table 4 in a BLBIO-5GC-4-H fermentation tank (Shanghai Bailun Biological Technology Co., Ltd.). After the fermentation, the ninhydrin colorimetry method was used to detect the L-lysine yield, and the dissolved oxygen (DO) (660 nm) was measured with a spectrophotometer. Each strain was repeated three times, and the results are shown in Table 5.

TABLE 3
Fermentation medium formula

| Composition | Formula | |
|---|---|---|
| Amylolytic sugar | 30 | g/L |
| Ammonium sulfate | 12 | g/L |
| Magnesium sulfate | 0.87 | g/L |
| Molasses | 20 | g/L |
| Acidified corn steep liquor | 3 | mL/L |
| Phosphoric acid | 0.4 | mL/L |
| Potassium chloride | 0.53 | g/L |
| Defoamer (2%) | 4 | mL/L |
| Ferrous sulfate | 120 | mg/L |
| Manganese sulfate | 120 | mg/L |
| Nicotinamide | 42 | mg/L |
| Calcium pantothenate | 6.3 | mg/L |
| Vitamin B1 | 6.3 | mg/L |
| Copper and zinc salt solution | 0.6 | g/L |
| Biotin | 0.88 | mg/L |

TABLE 4
Fermentation control process

| Correction DO100% | Temperature 37° C., air volume 4 L/min, rotating speed 1000 rpm, tank pressure | | |
|---|---|---|---|
| | 0 mpa, and calibration for 5 min | | |
| Inoculation amount | 10% | Culture temperature (° C.) | 37° C. |
| pH | pH 6.9 ± 0.05 | DO | 10-30% |

| | |
|---|---|
| Initial condition | Temperature 37° C., pH 6.9, tank pressure 0 Mpa, air volume 3 L/min, and |
| | rotating speed 550 rpm |
| Whole process | Whole process control: 1. When DO <30%, increasing the rotating speed |
| control | 750 rpm→800 rpm→air volume 4 L/min→850 rpm→950 rpm successively; 2. |
| | Fermenting for 6 h at the tank pressure of 0.01 Mpa; fermenting for 12 h at the |
| | tank pressure of 0.02 Mpa→0.03 Mpa→0.04 Mpa→0.05 Mpa |
| Residual sugar | 0.1-0.2% before F12 h; controlling residual sugar at 0.1-0.05% after F12 h |
| control | according to DO requirements |
| Ammonia nitrogen | 0.1-0.15 before F12 h; 0.15-0.25 between F12-F32 h; 0.1-0.15 after F32 h |
| control | |
| Materials added | 25% ammonia water, 70% concentrated sugar, 50% ammonium sulfide, 10% |
| | defoamer |
| Fermentation period | About 48 h |

TABLE 5
Experimental results of L-lysine fermentation

| | L-lysine | | Analysis on | |
|---|---|---|---|---|
| | yield | 0D | significant difference | |
| Strain | (g/100 ml) | (660 nm) | of L-lysine yield | |
| YP097158 | 18.9 | 37.3 | | |
| YPL-NCg12747-1 | 19.5 | 38.1 | P < 0.05 | Compared |
| YPL-NCg12747-2 | 19.2 | 37.8 | P > 0.05 | with |
| YPL-NCg12747-3 | 19.7 | 38.2 | P < 0.01 | TP097158 |
| YPL-NCg12747-4 | 19.4 | 38.2 | P < 0.05 | |
| YPL-NCg12747-5 | 19.9 | 38.3 | P < 0.01 | |
| YPL-NCg12747-6 | 18.0 | 36.8 | P < 0.01 | |
| ATCC13032 | 0.20 | 100.2 | | |
| L2747-1 | 0.35 | 92.3 | P < 0.01 | Compared |
| L2747-2 | 0.36 | 90.2 | P < 0.01 | with |
| L2747-3 | 0.42 | 91.5 | P < 0.01 | ATCC13032 |
| L2747-4 | 0.46 | 89.2 | P < 0.01 | |
| L2747-5 | 0.48 | 90.9 | P < 0.01 | |
| L2747-6 | 0.15 | 98.5 | P < 0.01 | |

The results are shown in Table 5. Point mutation NCg12747319F and overexpression of the Neg12747 gene coding region in the Corynebacterium glutamicum contributes to increase in the yield and the growth rate of L-lysine. However, when the gene is weakened or knocked out, accumulation of the L-lysine is not facilitated, and the growth rate of a strain can be reduced.

The present invention is described in detail above. For those skilled in the art, the present invention can be implemented in a wider range under equivalent parameters, concentrations and conditions without departing from the purpose and scope of the present invention and performing unnecessary experiments. Although special embodiments of the present invention have been shown, it should be understood that further improvements can be made to the present invention. In short, based on the principles of the present invention, this application is intended to include any changes in, uses of, or improvements to the present invention, including changes that depart from the scope disclosed in this application and are made using conventional technologies known in the art. Some essential features may be applied within the scope of the appended claims below.

The sequences 1-14 involved in the above embodiments are as follows:

Wild type ORF (CDS) sequence of NCgl2747 gene (nucleotide sequence 1314bp)
SEQ ID NO: 1
GTGACTACAGACAAGCGCAAAACCTCTAAGACCACCGACACCGCCAACAAGGCTGTGGGCGCGGATCAGGCAGC
GCGTCCCACTCGGCGAACAACTCGCCGCATCTTCGATCAGTCCGAGAAGATGAAGGACGTGCTGTACGAGATCC
GTGGCCCGGTGGCCGCGGAGGCGGAACGCATGGAGCTTGATGGGCATAACATCTTAAAGCTCAACACGGGAAAT
CCAGCCGTGTTCGGATTCGATGCCCCCGACGTGATTATGCGTGACATGATCGCCAACCTTCCAACTTCCCAAGG
GTATTCCACCTCCAAAGGCATTATTCCGGCCCGGCGAGCAGTGGTCACCCGCTACGAAGTTGTGCCCGGATTCC
CCCACTTCGATGTTGATGATGTGTTCTTAGGCAACGGTGTCTCAGAACTAATCACCATGACCACCCAAGCACTC
CTCAACGACGGCGATGAAGTTCTTATCCCCGCACCGGACTACCCACTGTGGACTGCCGCAACCTCCCTGGCTGG
TGGTAAGCCTGTGCACTACCTCTGTGATGAGGAAGATGACTGGAACCCATCCATCGAAGACATCAAGTCCAAAA
TCTCAGAGAAAACCAAAGCTATTGTGGTGATCAACCCCAACAACCCCACGGGAGCTGTCTACCCGCGCCGGGTG
TTGGAACAAATCGTCGAGATTGCACGCGAGCATGACCTGCTGATTTTGGCCGATGAAATCTACGACCGCATTCT
CTACGATGATGCCGAGCACATCAGCCTGGCAACCCTTGCACCAGATCTCCTTTGCATCACATACAACGGTCTAT
CCAAGGCATACCGCGTCGCAGGATACCGAGCTGGCTGGATGGTATTGACTGGACCAAAGCAATACGCACGTGGA
TTTATTGAGGGCCTCGAACTCCTCGCAGGCACTCGACTCTGCCCAAATGTCCCAGCTCAGCACGCTATTCAGGT
AGCTCTCGGTGGACGCCAGTCCATCTACGACCTCACTGGCGAACACGGCCGACTCCTGGAACAGCGCAACATGG
CATGGACGAAACTCAACGAAATCCCAGGTGTCAGCTGTGTGAAACCAATGGGACCTCTATACGCGTTCCCCAAG
CTCGACCCCAACGTGTACGAAATCCACGACGACACCCAACTCATGCTGGATCTTCTCCGTGCCGAGAAAATCCT
CATGGTTCAGGGCACTGGCTTCAACTGGCCACATCACGATCACTTCCGAGTGGTCACCCTGCCATGGGCATCCC
AGTTGGAAAACGCAATTGAGCGCCTGGGTAACTTCCTGTCCACTTACAAGCAGTAG
NCgl2747 protein sequence (i.e., amino acid sequence 438aa encoded
by sequence 1)
SEQ ID NO: 2
MTTDKRKTSKTTDTANKAVGADQAARPTRRTTRRIFDQSEKMKDVLYEIRGPVAAEAERMELDGHNILKLNTGN
PAVFGFDAPDVIMRDMIANLPTSQGYSTSKGTIPARRAVVTRYEVVPGPPHFDVDDYFLGNGVSELITMTTQAL
LNDGDEVLIPAPDYPLWTAATSLAGGKPVHVLCDEEDDWNPSIEDIKSKISEKTKAIVVINPNNPTGAVYPRRV
LEQIVEIAREHDLLILADEIYDRILYDDAEHISLATLAPDLLCITYNGLSKAYRVAGYRAGWMVLTGPKQYARG
FTEGLELLAGTRLCPNVPAQHAIQVALGGRQSTYDLTGEHGRLLEQRNMAWTKLNEIPGVSCVKPMGALYAFPK
LDPNVYEIHDDTQLMLDLLRAERILMVQGTGFNWPHHDHFRVVTLPWASQLENATERLGNFLSTYKQ
Gene mutant NCgl2747^{A955T}ORF (CDS) sequence (nucleotide sequence 1437bp)
SEQ ID NO: 3
GTGACTACAGACAAGCGCAAAACCTCTAAGACCACCGACACCGCCAACAAGGCTGTGGGCGCGGATCAGGCAGC
GCGTCCCACTCGGCGAACAACTCGCCGCATCTTCGATCAGTCGGAGAAGATGAAGGACGTGCTGTACGAGATCC
GTGGCCCGGTGGCCGCGGAGGCGGAACGCATGGACCTTGATGGGCATAACATCTTAAAGCTCAACACGGGAAAT
CCAGCCGTGTTCGGATTCGATGCCCCCGACGTGATTATGCGTGACATGATCGCCAACCTTCCAACTTCCCAAGG

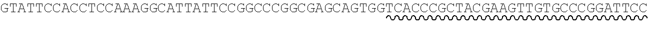

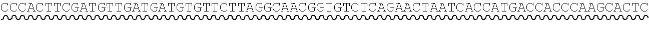

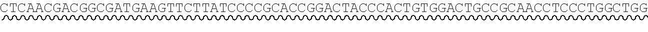

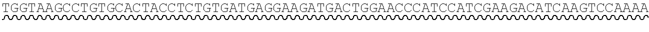

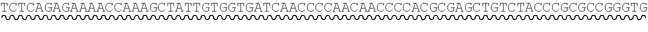

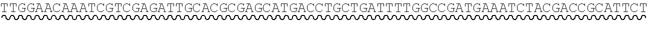

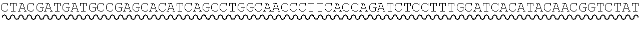

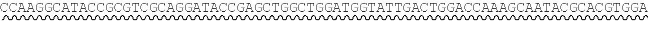

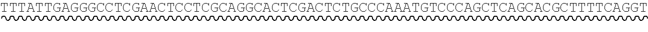

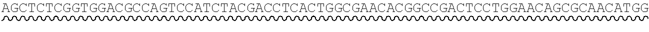

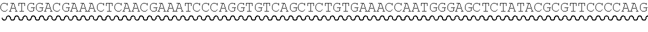

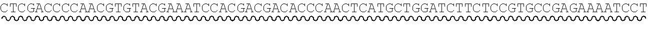

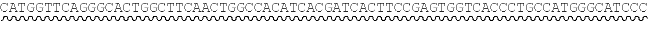

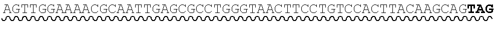

Gene mutant NCgl2747^{1319F} protein sequence (i.e., amino acid sequence
479aa encoded by sequence 3)
SEQ ID NO: 4
MTTKDRKTSKTTDTANKAVGADQAARPTRRTTRRIFDQSEKMKDVLYEIRGPVAAEAERMELDGHNILKLNTGN
PAVFGFDAPDVIMRDMIANLPTSQGYSTSKGIIPARRAVVTRYEVVPGFPHFDVDDVFLGNGVSELITMTTQAL
LNDGDEVLIPAPDYPLWTAATSLAGGKPVHYLCDEEDDWNSPIEDIKSKISEKTKAIVVINPNNPTGAVYPRRV
LEQIVEIAREHDLLILADEIYDRILYDDAEHISLATLAPDLLCITVNGLSKAYRVAGYRAGWMVLTGPKQYARG
FIEGLELLAGTRLCPNVPAQHAFQVALGGRQSIYDLTGEHGRLLEQRNMAWTKLNEIPGVSCVKPMGALYAFPK
LDPNVYEIHDDTQLMLDLLRAEKILMVQGTGFNWPHHDHFRVVTLPWASQLENAIERLGNFLSTYKQ
NCgl2747^{A955T}Up-Down (size of 1296bp)
SEQ ID NO: 5

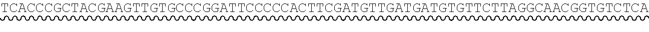

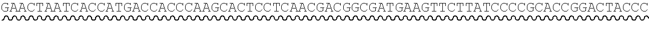

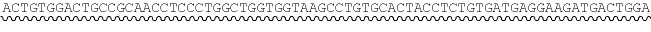

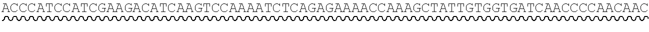

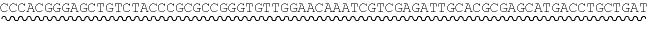

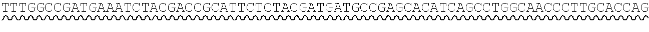

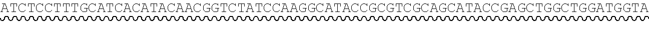

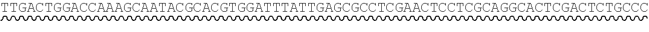

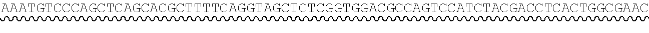

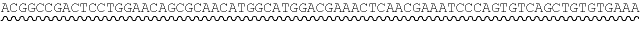

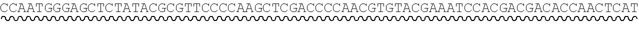

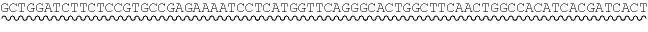

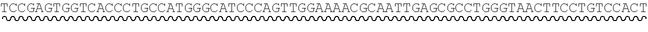

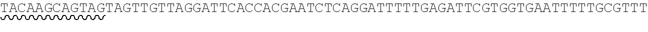

TCCAGTCAGGCTCCTGCAACTTTCGGACCGATTTCAGAGGGGCGGAGCTGGTTTGTGGTGGATCCTTGAAATGG
AACCTCGCAGGAAGCTTTCAGGAAGACCAAGTTGGGCCTAGGGGTGGCGGGATTGCAAAAATCCGTCCCCGGTT
CGCCATGAAATGCTGATTTTGATCGAATCTTTGCGCTAACTGTAGGGCGGGTTCAGGGGGTGAATGCACCACGA
GCAACCCGAAGGGTGCGAAGTGGGCATTCGTAGAACAA
Upstream homologous are sequence of genome-integrated P7/P8
NCgl2747 (763bp)
SEQ ID NO: 6
AATGCGTTCTGGACTGACGTGACCACCATGCAAGACGAGGTGGACCTAGTGATCACCAACCCGCCGTTTTCTCT
GTTCCGTGAGTTCCTGAGTTGGCTATTACACGGTGACGTGTTGTTTTCTATCATCGGTAACGCGAACGTAATCA
CATATCTAGGTGCCTGAATCTAGATTAAAACTATAAACATTATTTAAAACCATTGCCTTATTGGAGCATGCTGC
AAGCTTTTCGCGGTGGGCTTGCAACATCTTACATCAGAATGAATGATGTTAAACACCTAATAAGTTCAAGTAGT
TAAAAGGAAATATCAACAATGTTACGAAAAGCATCTATCACGCTAATGATATCCGTTACGCTCTTAACATGTGC
CTCTCCAGCACAGGCACTGTCATCACAAGCACTCTCGTCAGAAAGCTCCACTTCGCAGAGTGATTCTCCTACGC
AATTTGTTGCGAGTATAGCTGCGCCCTTCAATAAGAACTTAACCTATGAGCAGCGCAAAGCCATTCGTTTCGGA
TGCTATCTATCCTGACTGCTCCTTCGATACTATCGAACAAGATTTCTATGATCAAGCAATATCCAAAGCCCTCT
CAGTAATCAGTCTGGGAAGCTCCTAGAAGACTCGTAACGACCAAAAGCACCGAGAACAGATGCAACTCAAGCCC
ATTTACATCTGTTCTCGGTGCAC
Genome-integrated P9/P10 NCgl2747 and promoter sequence thereof (1645bp)
SEQ ID NO: 7
CAAAGCACTTGCTACTCAATCATGGTAAACAGCCTGGTCGCAGTCCTTCACGATTCAAACTTTGCCTTCCGCTA
CGCCTTCCACCTGATCATCATAGAAGACGGTGAAGTAACAGCAGCCGGAGATCCCACAGAGATCGTCACTGCGG
GACTGATCGAAGAAGTCTACAACGTCAAAGCCTGTGCATCCCAGACCCCGTGAACAGCAAACCGATGATCGTGC
CACTGGAAAGATCTTAGGCAGCCGTGGGATTACACCCTTTTAGAGCTAGAACAGTAAAAATTCACCCAATAGCT
TTCAACTACGCACACAAAGTGGCAACATTGAGCGGGTGACTACAGACAAGCGCAAAACCTCTAAGACCACCGAC
ACCGCCAACAAGGCTGTGGGCGCGGATCAGGCAGCGCGTCCCACTCGCCGAACAACTCGCCGCATCTTCGATCA
GTCGGAGAAGATGAAGGACGTGCTGTACGAGATCCGTGGCCCGGTGGCCGCGGAGGCGGAACGCATGGAGCTTG
ATGGGCATAACATCTTAAAGCTCAACACGGGAAATCCAGCCGTGTTCGGATTCGATGCCCCCGACGTGATTATG
CGTGACATGATCGCCAACCTTCCAACTTCCCAAGGGTATTCCACCTCCAAAGGCATTATTCCGGCCCGGCGAGC
AGTGGTCACCCGCTACGAAGTTGTGCCCGGATTCCCCCACTTCGATGTTGATGATGTGTTCTTAGGCAACGGTG
TCTCAGAACTAATCACCATGACCACCCAAGCACTCCTCAACGACGGCGATGAAGTTCTTATCCCCGCACCGGAC
TACCCACTGTGGACTGCCGCAACCTCCCTGGCTGGTGGTAAGCCTGTGCACTACCTCTGTGATGAGGAAGATGA
CTGGAACCCATCCATCGAAGACATCAAGTCCAAAATCTCAGAGAAAACCAAAGCTATTGTGGTGATCAACCCCA
ACAACCCCACGGGAGCTGTCTACCCGCGCCGGGTGTTGGAACAAATCGTCGAGATTGCACGCGAGCATGACCTG
CTGATTTTGGCCGATGAAATCTACGACCGCATTCTCTACGATGATGCCGAGCACATCAGCCTGGCAACCCTTGC
ACCAGATCTCCTTTGCATCACATACAACGGTCTATCCAAGGCATACCGCGTCGCAGGATACCGAGCTGGCTGGA
TGGTATTGACTGGACCAAAGCAATACGCACGTGGATTTATTGAGGGCCTCGAACTCCTCGCAGGCACTCGACTC
TGCCCAAATGTCCCAGCTCAGCACGCTATTCAGGTAGCTCTCGGTGGACGCCAGTCCATCTACGACCTCACTGG
CGAACACGGCCGACTCCTGGAACAGCCCAACATGGCATGGACGAAACTCAACGAAATCCCAGGTGTCAGCTGTG
TGAAACCAATGGGAGCTCTATACGCGTTCCCCAAGCTCGACCCCAACGTGTACGAAATCCACGACGACACCCAA
CTCATGCTGGATCTTCTCCGTGCCGAGAAAATCCTCATGGTTCAGGGCACTGGCTTCAACTGGCCACATCACGA
TCACTTCCGAGTGGTCACCCTGCCATGGGCATCCCAGTTGGAAAACGCAATTGAGCGCCTGGGTAACTTCCTGT
CCACTTACAAGCAGTAG
Genome-integrated P9/P10 NCgl2747^{A955T} and promoter sequence thereof (1645bp)
SEQ ID NO: 8
CAAAGCACTTGCTACTCAATCATGGTAAACAGCCTGGTCGCAGTCCTTCACGATTCAAACTTTGCCTTCCGCTA
CGCCTTCCACCTGATCATCATAGAAGACGGTGAAGTAACAGCAGCCGGAGATCCCACAGAGATCGTCACTGCGG
GACTGATCGAAGAAGTCTACAACGTCAAAGCCTGTGCATCCCAGACCCCGTGAACAGCAAACCGATGATCGTGC
CACTGGAAAGATCTTAGGCAGCCGTGGGATTACACCCTTTTAGAGCTAGAACAGTAAAAATTCACCCAATAGCT
TTCAACTACGCACACAAAGTGGCAACATTGAGCGGGTGACTACAGACAAGCGCAAAACCTCTAAGACCACCGAC
ACCGCCAACAAGGCTGTGGGCGCGGATCAGGCAGCGCGTCCCACTCGGCGAACAACTCGCCGCATCTTCGATCA
GTCGGAGAAGATGAAGGACGTGCTGTACGAGATCCGTGGCCCGGTGGCCGCGGAGGCGGAACGCATGGAGCTTG
ATGGGCATAACATCTTAAAGCTCAACACGGGAAATCCAGCCGTGTTCGGATTCGATGCCCCCGACGTGATTATG
CGTGACATGATCGCCAACCTTCCAACTTCCCAAGGGTATTCCACCTCCAAAGGCATTATTCCGGCCCGGCGAGC
AGTGGTCACCCGCTACGAAGTTGTGCCCGGATTCCCCCACTTCGATGTTGATGATGTGTTCTTAGGCAACGGTG
TCTCAGAACTAATCACCATGACCACCCAAGCACTCCTCAACGACGGCGATGAAGTTCTTATCCCCGCACCGGAC
TACCCACTGTGGACTGCCGCAACCTCCCTGGCTGGTGGTAAGCCTGTGCACTACCTCTGTGATGAGGAAGATGA
CTGGAACCCATCCATCGAAGACATCAAGTCCAAAATCTCAGAGAAAACCAAAGCTATTGTGGTGATCAACCCCA
ACAACCCCACGGGAGCTGTCTACCCGCGCCGGGTGTTGGAACAAATCGTCGAGATTGCACGCGAGCATGACCTG
CTGATTTTGGCCGATGAAATCTACGACCGCATTCTCTACGATGATGCCGAGCACATCAGCCTGGCAACCCTTGC
ACCAGATCTCCTTTGCATCACATACAACGGTCTATCCAAGGCATACCGCGTCGCAGGATACCGAGCTGGCTGGA
TGGTATTGACTGGACCAAAGCAATACGCACGTGGATTTATTGAGGGCCTCGAACTCCTCGCAGGCACTCGACTC
TGCCCAAATGTCCCAGCTCAGCACGCTTTTCAGGTAGCTCTCGGTGGACGCCAGTCCATCTACGACCTCACTGG
CGAACACGGCCGACTCCTGGAACAGCGCAACATGGCATGGACGAAACTCAACGAAATCCCAGGTGTCAGCTGTG
TGAAACCAATGGGAGCTCTATACGCGTTCCCCAAGCTCGACCCCAACGTGTACGAAATCCACGACGACACCCAA
CTCATGCTGGATCTTCTCCGTGCCGAGAAAATCCTCATGGTTCAGGGCACTGGCTTCAACTGGCCACATCACGA
TCACTTCCGAGTGGTCACCCTGCCATGGGCATCCCAGTTGGAAAACGCAATTGAGCGCCTGGGTAACTTCCTGT
CCACTTAGAAGCAGTAG
Downstream homologous arm sequence of genome-integrated P11/P12
NCgl2747 (596bp)
SEQ ID NO: 9
CAGATGGCGCAATTAAATCAAGATCTCAGAACTCATTTTTCAATCTCTTCTTTTAGGGCACCCGTCATCAATTG
AGCTATCGGCCATTCATTAAAAACTGCGCGTCGATCAACCGAAAGCTGAGTAAGCAAAAAATTCGCCTCTTCTC
TATCGTCAAGAAGCAAAGCGCAACCAATCATAATCTCGCTATAGTTGTCTGGGTGCATTGTTTGATCAAGATTA
TGCCTAAAAGATCGAATTTCTGTTCTTTGTGAATCCAGTAATCCAGTTTGGCGATACAAAATTTGCCAACCATT
AAGCCTGTAAATTGGCGACTCTTCTCGATCTCGTTCCACGAGCCAATCATTGAGAGCTTGAGCTGCTATAAGAA
ATGCAGTTTTCCTGCTCTGCTCAGAATCAGCTGCCTTAATTAGGCGAAGCACCATCCATGTGGCGAGATTATCA
ACATCAACAGTTTCTTCAAGCGCTGAATAGAAATCAACAAGCTTATCCAAATGCAGATTCAATACTGATGGAAC
CCATCGATTATCAATGAGCTCGTACGGCGTCACTCGCTCGATTGAGGAGTCGCTCCGATCCGTTGAAGGTGTCA
TAGC
Identification primers P13 and P14 amplified fragment (size of 1959bp)
SEQ ID NO: 10
TCCAAGGAAGATACACGCCCGAAGAAGCTCCGTTAATGGACGCTCTTCTATTAAGCCTTTTCTGATGGATACAA
GCAGAGAATCAGTCATGCTAAATATCATAATCCATCAAACATTTGATCGGTGTCAATCAATGCGTTCTGGACTG
AGGTGACCACCATGCAAGACGAGGTGGACCTAGTGATCACCAACCCGCCGTTTTCTCTGTTCCGTGAGTTCCTG
AGTTGGCTATTACACGGTGACGTGTTGTTTTCTATCATCGGTAACGCGAACGTAATCACATATCTAGGTGCCTG
AATCTAGATTAAAACTATAAACATTATTTAAAACCATTGCCTTATTGGAGCATGCTGCAAGCTTTTCGCGGTGG
GCTTGCAACATCTTACATCAGAATGAATGATGTTAAACACCTAATAAGTTCAAGTAGTTAAAAGGAAATATCAA
CAATGTTACGAAAAGCATCTATCACGCTAATGATATCCGTTACGCTCTTAACATGTGCCTCTCCAGCACAGGCA
CTGTCATCACAAGCACTCTCGTCAGAAAGCTCCACTTCGCAGAGTGATTCTCCTACGCAATTTGTTGCGAGTAT
AGCTGCGCCCTTCAATAAGAACTTAACCTATGAGCAGCGCAAAGCCATTCGTTTCGGACCTACGACAGAACAAG
AAGCAGAACAATGCTTGGCAAAGTATGGTCGCGATGGCGAGGGGCCATGGCCTGCTATTGCTATCTATCCTGAC
TGCTCCTTCGATACTATCGAACAAGATTTCTATGATCAAGCAATATCCAAAGCCCTCTCAGTAATCAGTCTGGG
AAGCTCCTAGAAGACTCGTAACGACCAAAAGCACCGAGAACAGATGCAACTCAAGCCCATTTACATCTGTTCTC
GGTGCACCAAAGCACTTGCTACTCAATCATGGTAAACAGCCTGGTCGCAGTCCTTCACGATTCAAACTTTGCCT
TCCGCTACGCCTTCCACCTGATCATCATAGAAGACGGTGAAGTAACAGCAGCCGGAGATCCCACAGAGATCGTC
ACTGCGGGACTGATCGAAGAAGTCTACAACGTCAAAGCCTGTGCATCCCAGACCCCGTGAACAGCAAACCGATG
ATCGTGCCACTGGAAAGATCTTAGGCAGCCGTGGGATTACACCCTTTTAGAGCTAGAACAGTAAAAATTCACCC
AATAGCTTTCAACTACGCACACAAAGTGGCAACATTGAGCGGGTGACTACAGACAAGCGCAAAACCTCTAAGAC
CACCGACACCGCCAACAAGGCTGTGGGCGCGGATCAGGCAGCGCGTCCCACTCGGCGAACAACTCGCCGCATCT
TCGATCAGTCGGAGAAGATGAAGGACGTGCTGTACGAGATCCGTGGCCCGGTGGCCGCGGAGGCGGAACGCATG
GAGCTTGATGGGCATAACATCTTAAAGCTCAACACGGGAAATCCAGCCGTGTTCGGATTCGATGCCCCCGACGT
GATTATGCGTGACATGATCGCCAACCTTCCAACTTCCCAAGGGTATTCCACCTCCAAAGGCATTATTCCGGCCC
GGCGAGCAGTGGTCACCCGCTACGAAGTTGTGCCCGGATTCCCCCACTTCGATGTTGATGATGTGTTCTTAGGC
AACGGTGTCTCAGAACTAATCACCATGACCACCCAAGCACTCCTCAACGACGGCGATGAAGTTCTTATCCCCGC
ACCGGACTACCCACTGTGGACTGCCGCAACCTCCCTGGCTCCTCCTAAGCCTGTGCACTACCTCTGTGATGAGG
AAGATGACTGGAACCCATCCATCGAAGACATCAAGTCCAAAATCTCAGAGAAAACCAAAGCTATTGTGGTGATC
AACCCCAACAACCCCACGGGAGCTGTCTACCCGCGCCGGGTGTTGGAACAAATCGTCGAGATTGCACGCGAGCA
TGACCTGCTGATTTTGGCCGATGAAATCTACGACC
Identification primers P15 and P16 amplified fragment (size of 1600bp)
SEQ ID NO: 11
CAACGGTGTCTCAGAACTAATCACCATGACCACCCAAGCACTCCTCAACGACGGCGATGAAGTTCTTATCCCCG
CACCGGACTACCCACTGTGGACTGCCGCAACCTCCCTGGCTGGTGGTAAGCCTGTGCACTACCTCTGTGATGAG
GAAGATGACTGGAACCCATCCATCGAAGACATCAAGTCCAAAATCTCAGAGAAAACCAAAGCTATTGTGGTGAT
CAACCCCAACAACCCCACGGGAGCTGTCTACCCGCGCCGGGTGTTGGAACAAATCGTCGAGATTGCACGCGAGC
ATGACCTGCTGATTTTGGCCGATGAAATCTACGACCGCATTCTCTACGATGATGCCGAGCACATCAGCCTGGCA
ACCCTTGCACCAGATCTCCTTTGCATCACATACAACGGTCTATCCAAGGCATACCGCGTCGCAGGATACCGAGC
TGGCTGGATGGTATTGACTGGACCAAAGCAATACGCACGTGGATTTATTGAGGGCCTCGAACTCCTCGCAGGCA
CTCGACTCTGCCCAAATGTCCCAGCTCAGCACGCTATTCAGGTAGCTCTCGGTGGACGCCAGTCCATCTACGAC
CTCACTGGCGAACACGGCCGACTCCTGGAACAGCGCAACATGGCATGGACGAAACTCAACGAAATCCCAGGTGT
CAGCTGTGTGAAACCAATGGGAGCTCTATACGCGTTCCCCAAGCTCGACCCCAACGTGTACGAAATCCACGACG
ACACCCAACTCATGCTGGATCTTCTCCGTGCCGAGAAAATCCTCATGGTTCAGGGCACTGGCTTCAACTGGCCA
CATCACGATCACTTCCGAGTGGTCACCCTGCCATGGGCATCCCAGTTGGAAAACGCAATTGAGCGCCTGGGTAA
CTTCCTGTCCACTTACAAGCAGTAGCAGATGGCGCAATTAAATCAAGATCTCAGAACTCATTTTTCAATCTCTT
CTTTTAGGGCACCCGTCATCAATTGAGCTATCGGCCATTCATTAAAAACTGCGCGTCGATCAACCGAAAGCTGA
GTAAGCAAAAAATTCGCCTCTTCTCTATCGTCAAGAAGCAAAGCGCAACCAATCATAATCTCGCTATAGTTGTC
TGGGTGCATTGTTTGATCAAGATTATGCCTAAAAGATCGAATTTCTGTTCTTTGTGAATCCAGTAATCCAGTTT
GGCGATACAAAATTTGCCAACCATTAAGCCTGTAAATTGGCGACTCTTCTCGATCTCGTTCCACGAGCCAATCA
TTGAGAGCTTGAGCTGCTATAAGAAATGCAGTTTTCCTGCTCTGCTCAGAATCAGCTGCCTTAATTAGGCGAAG
CACCATCCATGTGGCGAGATTATCAACATCAACAGTTTCTTCAAGCGCTGAATAGAAATCAACAAGCTTATCCA
AATGCAGATTCAATACTGATGGAAGCCATCGATTATCAATGAGCTCGTACGGCGTCACTCGCTCGATTGAGGAG
TCGCTCCGATCCGTTGAAGGTGTCATAGCAAAATGATGACGCTTGGAAATTACTTCATGATAAGTCCACTCCCC
TGCAACATCACCAGGGTGAGCTATCAACGCAAGATTCCAACGACCA
NCgl2747 overexpressed by P17 and P18 amplified pXMJ19 plasmid and
promoter sequence thereof (size of 1715bp)
SEQ ID NO: 12

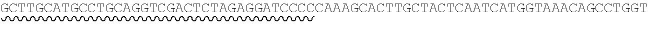

CGCAGTCCTTCACGATTCAAACTTTGCCTTCCGCTACGCCTTCCACCTGATCATCATAGAAGACGGTGAAGTAA
CAGCAGCCGGAGATCCCACAGAGATCGTCACTGCGGGACTGATCCAAGAAGTCTACAACGTCAAACCCTGTGCA
TCCCAGACCCCGTGAACAGCAAACCGATGATCGTGCCACTGGAAAGATCTTAGGCAGCCGTGGGATTACACCCT
TTTAGAGCTAGAACAGTAAAAATTCACCCAATAGCTTTCAACTACGCACACAAAGTGGCAACATTGAGCGGGTG
ACTACAGACAAGCGCAAAACCTCTAAGACCACCGACACCGCCAACAAGGCTGTGGGCGCGGATCAGGCAGCGCG
TCCCACTCGGCGAACAACTCGCCGCATCTTCGATCAGTCGGAGAAGATGAAGGACGTGCTGTACGAGATCCGTG
GCCCGGTGGCCGCGGAGGCGGAACGCATGGAGCTTGATGGGCATAACATCTTAAAGCTCAACACGGGAAATCCA
GCCGTGTTCGGATTCGATGCCCCCGACGTGATTATGCGTGACATGATCGCCAACCTTCCAACTTCCCAAGGGTA
TTCCACCTCCAAAGGCATTATTCCGGCCCGGCGAGCAGTGGTCACCCGCTACGAAGTTGTGCCCGGATTCCCCC
ACTTCGATGTTGATGATGTGTTCTTAGGCAACGGTGTCTCAGAACTAATCACCATGACCACCCAAGCACTCCTC
AACGACGGCGATGAAGTTCTTATCCCCGCACCGGACTACCCACTGTGGACTGCCGCAACCTCCCTGGCTGGTGG
TAAGCCTGTGCACTACCTCTGTGATGAGGAAGATGACTGGAACCCATCCATCGAAGACATCAAGTCCAAAATCT
CAGAGAAAACCAAAGCTATTGTGGTGATCAACCCCAACAACCCCACGGGAGCTGTCTACCCGCGCCGGGTGTTG
GAACAAATCGTCGAGATTGCACGCGAGCATGACCTGCTGATTTTGGCCGATGAAATCTACGACCGCATTCTCTA
CGATGATGCCGAGCACATCAGCCTGGCAACCCTTGCACCAGATCTCCTTTGCATCACATACAACGGTCTATCCA
AGGCATACCGCGTCGCAGGATACCGAGCTGGCTGGATGGTATTGACTGGACCAAAGCAATACGCACGTGGATTT
ATTGAGGGCCTCGAACTCCTCGCAGGCACTCGACTCTGCCCAAATGTCCCAGCTCAGCACGCTATTCAGGTAGC
TCTCGGTGGACGCCAGTCCATCTACGACCTCACTGGCGAACACGGCCGACTCCTGGAACAGCGCAACATGGCAT
GGACGAAACTCAACGAAATCCCAGGTGTCAGCTGTGTGAAACCAATGGGAGCTCTATACGCGTTCCCCAAGCTC
GACCCCAACGTGTACGAAATCCACGACGACACCCAACTCATGCTGGATCTTCTCCGTGCCGAGAAAATCCTCAT
GGTTCAGGGCACTGGCTTCAACTGGCCACATCACGATCACTTCCGAGTGGTCACCCTGCCATGGGCATCCCAGT

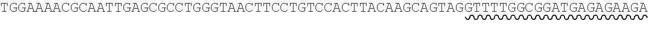

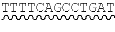

NCgl2747^{A955T} overexpressed by P17 and P18 amplified pXMJ19 plasmid
and promoter sequence thereof (size of 1715bp)
SEQ ID NO: 13

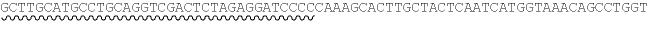

CGCAGTCCTTCACGATTCAAACTTTGCCTTCCGCTACGCCTTCCACCTGATCATCATAGAAGACGGTGAAGTAA
CAGCAGCCGGAGATCCCACAGAGATCGTCACTGCGGGACTGATCGAAGAAGTCTACAACGTCAAAGCCTGTGCA
TCCCAGACCCCGTGAACAGCAAACCGATGATCGTGCCACTGGAAAGATCTTAGGCAGCCGTGGGATTACACCCT
TTTAGAGCTAGAACAGTAAAAATTCACCCAATAGCTTTCAACTACGCACACAAAGTGGCAACATTGAGCGGGTG
ACTACAGACAAGCGCAAAACCTCTAAGACCACCGACACCGCCAACAAGGCTGTGGGCGCGGATCAGGCAGCGCG
TCCCACTCGGCGAACAACTCGCCGCATCTTCGATCAGTCGGAGAAGATGAAGGACGTGCTGTACGAGATCCGTG
GCCCGGTGGCCGCGGAGGCGGAACGCATGGAGCTTGATGGGCATAACATCTTAAAGCTCAACACGGGAAATCCA
GCCGTGTTCGGATTCGATGCCCCCGACGTGATTATGCGTGACATGATCGCCAACCTTCCAACTTCCCAAGGGTA
TTCCACCTCCAAAGGCATTATTCCGGCCCGGCGAGCAGTGGTCACCCGCTACGAAGTTGTGCCCGGATTCCCCC
ACTTCGATGTTGATGATGTGTTCTTAGGCAACGGTGTCTCAGAACTAATCACCATGACCACCCAAGCACTCCTC
AACGACGGCGATGAAGTTCTTATCCCCGCACCGGACTACCCACTGTGGACTGCCGCAACCTCCCTGGCTGGTGG
TAAGCCTGTGCACTACCTCTGTGATGAGGAAGATGACTGGAACCCATCCATCGAAGACATCAAGTCCAAAATCT
CAGAGAAAACCAAAGCTATTGTGGTGATCAACCCCAACAACCCCACGGGAGCTGTCTACCCGCGCCGGGTGTTG
GAACAAATCGTCGAGATTGCACGCGAGCATGACCTGCTGATTTTGGCCGATGAAATCTACGACCGCATTCTCTA
CGATGATGCCGAGCACATCAGCCTGGCAACCCTTGCACCAGATCTCCTTTGCATCACATACAACGGTCTATCCA
AGGCATACCGCGTCGCAGGATACCGAGCTGGCTGGATGGTATTGACTGGACCAAAGCAATACGCACGTGGATTT
ATTGAGGGCCTCGAACTCCTCGCAGGCACTCGACTCTGCCCAAATGTCCCAGCTCAGCACGCTTTTCAGGTAGC
TCTCGGTGGACGCCAGTCCATCTACGACCTCACTGGCGAACACGGCCGACTCCTGGAACAGCGCAACATGGCAT
GGACGAAACTCAACGAAATCCCAGGTGTCAGCTGTGTGAAACCAATGGGAGCTCTATACGCGTTCCCCAAGCTC
GACCCCAACGTGTACGAAATCCACGACGACACCCAACTCATGCTGGATCTTCTCCGTGCCGAGAAAATCCTCAT
GGTTCAGGGCACTGGCTTCAACTGGCCACATCACGATCACTTCCGAGTGGTCACCCTGCCATGGGCATCCCAGT

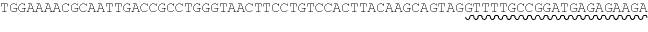

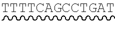

Homologous arm sequence of knockout NCgl2747 (size of 1400bp)
SEQ ID NO: 14
GCAGCAAGTAGCCTGGGTATTCGCCACGGACGTGAATATTGCCGAAGGCATAGTCCTCGTGGGACTTTTGCTGG
GCGGTAAGTTCATCGCGTGGGTCCGGGGTAATGCCATAACGAGGAACGGCAATAATCATGCAACCGATCTGGTT
TTGTGGGTCGATCTCATGAGCAATCTTAGTTGCCAAAGCACTTGCTACTCAATCATGGTAAACAGCCTGGTCGC
AGTCCTTCACGATTCAAACTTTGCCTTCCGCTACGCCTTCCACCTGATCATCATAGAAGACGGTGAAGTAACAG
CAGCCGGAGATCCCACAGAGATCGTCACTGCGGGACTGATCGAAGAAGTCTACAACGTCAAAGCCTGTGCATCC
CAGACCCCGTGAACAGCAAACCGATGATCGTGCCACTGGAAAGATCTTAGGCAGCCGTGGGATTACACCCTTTT
AGAGCTAGAACAGTAAAAATTCACCCAATAGCTTTCAACTACGCACACAAAGTGGCAACATTGAGCGGGTGACT
ACAGACAAGCGCAAAACCTCTAAGACCACCGACACCGCCAACAAGGCTGTGGGCGCGGATCAGGCAGCGCGTCC
CACTCGGCGAACAACTCGCCGCATCTTCGATCAGTCGGAGAAGATGAAGGACGTGGGTAACTTCCTGTCCACTT
ACAAGCAGTAGTAGTTGTTAGGATTCACCACGAATCTCAGGATTTTTGAGATTCGTGGTGAATTTTTGCGTTTT
CCAGTCAGGCTCCTGCAACTTTCGGACCGATTTCAGAGGGGCGGAGCTGGTTTGTGGTGGATCCTTGAAATGGA
ACCTCGCAGGAAGCTTTCAGGAAGACCAAGTTGGGCCTAGGGGTGGCGGGATTGCAAAAATCCGTCCCCGGTTC
GCCATGAAATGCTGATTTTGATCGAATCTTTGCGCTAACTGTAGGGCGGGTTCAGGGGGTGAATGCACCACGAG
CAACCCGAAGGGTGCGAAGTGGGCATTCGTAGAACAATCCCAGAGGAAAGCCGTACGGCTTTCCTCGACATGAT
CAATCAAGGTATGTCAGGTCTTGCTGCGTCTACAGCGGTCGGGGTCAGTGAATTCACCGGGCGAAAGTGGGCGA
AGGCCGCCGGGGTGAAACTGACCCGCGGCCCGCGAGGTGGCAATGCTTTTGACACCGCCGAGAAACTTGAGATT
GCAGCCAGCATGCTAGAGAAAGGATGCCTACCCCGAGAAATCGGCGAGTATGTCGGCATGACTCGGGCCAATAT
ATCCCTATGGCGCAAACAAGGCCCAGACAAGCTTCGCCAACGCGCAGCCACCTTGCGCACCGGCAAGCGAGCAG
CTGAATTCATCCACGCCCCGGTGATGGGCCCTTATTATGGGCCACGCACACTCCATCAAGTGTTGCGT

### INDUSTRIAL APPLICATION

According to the present invention, point mutation NCg127471319F and overexpression of the NCg12747 gene coding region in the Corynebacterium glutamicum contributes to increase in the yield and the growth rate of L-lysine. However, when the gene is weakened or knocked out, accumulation of the L-lysine is not facilitated, and the growth rate of a strain can be reduced. It shows that NCg127471319F and its encoding gene of the present invention can be used to prepare the L-lysine and have good application prospects.

What is claimed is:

1. A protein, wherein the protein is the following A1) or A2):
A1) a protein comprising the amino acid sequence of SEQ ID NO: 4;
A2) a fusion protein comprising a tag connected to an N-terminal or/and a C-terminal of A1).

2. A biological material related to the protein according to claim 1, wherein the biological material is one of the following B1) to B4):
B1) a nucleic acid molecule encoding the protein;
B2) an expression cassette comprising the nucleic acid molecule in B1);
B3) a recombinant vector comprising the nucleic acid molecule in B1), or a recombinant vector comprising the expression cassette in B2);
B4) a recombinant microorganism comprising the nucleic acid molecule in B1), or a recombinant microorganism comprising the expression cassette in B2), or a recombinant microorganism comprising the recombinant vector in B3).

3. The biological material according to claim 2, wherein the nucleic acid molecule in B1) is the following b11) or b12) or b13):
b11) a DNA molecule shown in SEQ ID NO: 3;
b12) a DNA molecule having 75% or more identity with a nucleotide sequence defined by b11) and used for encoding the protein;
b13) a genomic DNA molecule hybridizing with the nucleotide sequence defined by b11) or a nucleotide sequence defined by b12) under stringent conditions and used for encoding the protein.

4. The biological material according to claim 2, wherein the expression cassette in B2) is the DNA molecule shown in SEQ ID NO: 8;
the recombinant microorganism in B4) is a recombinant microorganism obtained by replacing an NCg12747 gene in a microorganism containing the NCg12747 gene shown in SEQ ID NO: 1 with the nucleic acid molecule in B1), or introducing the nucleic acid molecule in B1) into the microorganism and expressing the nucleic acid molecule.

5. A method for preparing L-lysine, comprising: expressing the protein according to claim 1 in a recipient biological cell, or increasing a content or activity of the protein in the recipient biological cell, or increasing a content or activity of the protein shown in SEQ ID NO: 2 in the recipient biological cell to obtain a recombinant biological cell; and culturing the recombinant biological cell to obtain the L-lysine.

6. The method according to claim 5, wherein the recombinant biological cell is yeast, bacterium, algae, fungi, a plant cell, or an animal cell configured for synthesizing the L-lysine.

7. The method according to claim 6, wherein the bacterium is Corynebacterium glutamicum.

8. The method according to claim 5, wherein the method is realized by introducing an encoding gene of the protein into the recipient biological cell and expressing the encoding gene, or introducing an encoding gene of the protein shown in SEQ ID NO: 2 into the recipient biological cell and expressing the encoding gene;
or, the recipient biological cell contains the DNA molecule shown in SEQ ID NO: 1, and the method is realized by replacing the DNA molecule shown in SEQ ID NO: 1 in the recipient biological cell with the DNA molecule shown in SEQ ID NO: 3.

9. The method according to claim 5, wherein the recombinant biological cell is cultured using a culture medium configured for growing the recombinant biological cell;
and/or, the recombinant biological cell is cultured using conditions for a growth of the recombinant biological cell.

10. The method according to claim 9, wherein the method is realized by introducing an encoding gene of the protein into the recipient biological cell and expressing the encoding gene, or introducing an encoding gene of the protein shown in SEQ ID NO: 2 into the recipient biological cell and expressing the encoding gene;
or, the recipient biological cell contains the DNA molecule shown in SEQ ID NO: 1, and the method is realized by replacing the DNA molecule shown in SEQ ID NO: 1 in the recipient biological cell with the DNA molecule shown in SEQ ID-No-SEQ ID NO: 3.

11. A product for preparing L-lysine, comprising the protein according to claim 1 or a biological material related to the protein, wherein the biological material is one of the following B1) to B4):
B1) a nucleic acid molecule encoding the protein;
B2) an expression cassette comprising the nucleic acid molecule in B1);
B3) a recombinant vector comprising the nucleic acid molecule in B1), or a recombinant vector comprising the expression cassette in B2);
B4) a recombinant microorganism comprising the nucleic acid molecule in B1), or a recombinant microorganism comprising the expression cassette in B2), or a recombinant microorganism comprising the recombinant vector in B3).

12. The biological material according to claim 3, wherein the expression cassette in B2) is the DNA molecule shown in SEQ ID NO: 8;
the recombinant microorganism in B4) is a recombinant microorganism obtained by replacing an NCgl2747 gene in a microorganism containing the NCg12747 gene shown in SEQ ID NO: 1 with the nucleic acid molecule in B1), or introducing the nucleic acid molecule in B1) into the microorganism and expressing the nucleic acid molecule.

13. The method according to claim 6, wherein the method is realized by introducing an encoding gene of the protein into the recipient biological cell and expressing the encoding gene, or introducing an encoding gene of the protein shown in SEQ ID NO: 2 into the recipient biological cell and expressing the encoding gene;
or, the recipient biological cell contains the DNA molecule shown in SEQ ID NO: 1, and the method is realized by replacing the DNA molecule shown in SEQ ID NO: 1 in the recipient biological cell with the DNA molecule shown in SEQ ID NO: 3.

14. The method according to claim 7, wherein the method is realized by introducing an encoding gene of the protein into the recipient biological cell and expressing the encoding gene, or introducing an encoding gene of the protein shown in SEQ ID NO: 2 into the recipient biological cell and expressing the encoding gene;
or, the recipient biological cell contains the DNA molecule shown in SEQ ID NO: 1, and the method is realized by replacing the DNA molecule shown in SEQ ID NO: 1 in the recipient biological cell with the DNA molecule shown in SEQ ID NO: 3.

15. The method according to claim 6, wherein the recombinant biological cell is cultured using a culture medium configured for growing the recombinant biological cell;
and/or, the recombinant biological cell is cultured using conditions for a growth of the recombinant biological cell.

16. The method according to claim 7, wherein the recombinant biological cell is cultured using a culture medium configured for growing the recombinant biological cell;
and/or, the recombinant biological cell is cultured using conditions for a growth of the recombinant biological cell.

17. The product according to claim 11, wherein the nucleic acid molecule in B1) is the following b11) or b12) or b13):
b11) a DNA molecule shown in SEQ ID NO: 3;
b12) a DNA molecule having 75% or more identity with a nucleotide sequence defined by b11) and used for encoding the protein;
b13) a genomic DNA molecule hybridizing with the nucleotide sequence defined by b11) or a nucleotide sequence defined by b12) under stringent conditions and used for encoding the protein.

18. The product according to claim 11, wherein the expression cassette in B2) is the DNA molecule shown in SEQ ID NO: 8;
the recombinant microorganism in B4) is a recombinant microorganism obtained by replacing an NCg12747 gene in a microorganism containing the NCg12747 gene shown in SEQ ID NO: 1 with the nucleic acid molecule in B1), or introducing the nucleic acid molecule in B1) into the microorganism and expressing the nucleic acid molecule.


SEQUENCE LISTING (43 sequences)

SEQ ID NO: 1 - DNA, 1314 bp, Corynebacterium glutamicum
  source 1..1314: mol_type = unassigned DNA
gtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgctattcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtag

SEQ ID NO: 2 - protein, 437 aa, Corynebacterium glutamicum
MTTDKRKTSKTTDTANKAVGADQAARPTRRTTRRIFDQSEKMKDVLYEIRGPVAAEAERMELDGHNILKLNTGNPAVFGFDAPDVIMRDMIANLPTSQGYSTSKGIIPARRAVVTRYEVVPGFPHFDVDDVFLGNGVSELITMTTQALLNDGDEVLIPAPDYPLWTAATSLAGGKPVHYLCDEEDDWNPSIEDIKSKISEKTKAIVVINPNNPTGAVYPRRVLEQIVEIAREHDLLILADEIYDRILYDDAEHISLATLAPDLLCITYNGLSKAYRVAGYRAGWMVLTGPKQYARGFIEGLELLAGTRLCPNVPAQHAIQVALGGRQSIYDLTGEHGRLLEQRNMAWTKLNEIPGVSCVKPMGALYAFPKLDPNVYEIHDDTQLMLDLLRAEKILMVQGTGFNWPHHDHFRVVTLPWASQLENAIERLGNFLSTYKQ

SEQ ID NO: 3 - DNA, 1314 bp, synthetic construct
  source 1..1314: mol_type = other DNA
gtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgcttttcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtag

SEQ ID NO: 4 - protein, 437 aa, synthetic construct
MTTDKRKTSKTTDTANKAVGADQAARPTRRTTRRIFDQSEKMKDVLYEIRGPVAAEAERMELDGHNILKLNTGNPAVFGFDAPDVIMRDMIANLPTSQGYSTSKGIIPARRAVVTRYEVVPGFPHFDVDDVFLGNGVSELITMTTQALLNDGDEVLIPAPDYPLWTAATSLAGGKPVHYLCDEEDDWNPSIEDIKSKISEKTKAIVVINPNNPTGAVYPRRVLEQIVEIAREHDLLILADEIYDRILYDDAEHISLATLAPDLLCITYNGLSKAYRVAGYRAGWMVLTGPKQYARGFIEGLELLAGTRLCPNVPAQHAFQVALGGRQSIYDLTGEHGRLLEQRNMAWTKLNEIPGVSCVKPMGALYAFPKLDPNVYEIHDDTQLMLDLLRAEKILMVQGTGFNWPHHDHFRVVTLPWASQLENAIERLGNFLSTYKQ

SEQ ID NO: 5 - DNA, 1296 bp, synthetic construct
  source 1..1296: mol_type = other DNA
tcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgcttttcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtagtagttgttaggattcaccacgaatctcaggatttttgagattcgtggtgaatttttgcgttttccagtcaggctcctgcaactttcggaccgatttcagaggggcggagctggtttgtggtggatccttgaaatggaacctcgcaggaagctttcaggaagaccaagttgggcctaggggtggcgggattgcaaaaatccgtccccggttcgccatgaaatgctgattttgatcgaatctttgcgctaactgtagggcgggttcagggggtgaatgcaccacgagcaacccgaagggtgcgaagtgggcattcgtagaacaa

SEQ ID NO: 6 - DNA, 763 bp, synthetic construct
  source 1..763: mol_type = other DNA
aatgcgttctggactgaggtgaccaccatgcaagacgaggtggacctagtgatcaccaacccgccgttttctctgttccgtgagttcctgagttggctattacacggtgacgtgttgttttctatcatcggtaacgcgaacgtaatcacatatctaggtgcctgaatctagattaaaactataaacattatttaaaaccattgccttattggagcatgctgcaagcttttcgcggtgggcttgcaacatcttacatcagaatgaatgatgttaaacacctaataagttcaagtagttaaaaggaaatatcaacaatgttacgaaaagcatctatcacgctaatgatatccgttacgctcttaacatgtgcctctccagcacaggcactgtcatcacaagcactctcgtcagaaagctccacttcgcagagtgattctcctacgcaatttgttgcgagtatagctgcgcccttcaataagaacttaacctatgagcagcgcaaagccattcgtttcggacctacgacagaacaagaagcagaacaatgcttggcaaagtatggtcgcgatggcgaggggccatggcctgctattgctatctatcctgactgctccttcgatactatcgaacaagatttctatgatcaagcaatatccaaagccctctcagtaatcagtctgggaagctcctagaagactcgtaacgaccaaaagcaccgagaacagatgcaactcaagcccatttacatctgttctcggtgcac

SEQ ID NO: 7 - DNA, 1645 bp, synthetic construct
  source 1..1645: mol_type = other DNA
caaagcacttgctactcaatcatggtaaacagcctggtcgcagtccttcacgattcaaactttgccttccgctacgccttccacctgatcatcatagaagacggtgaagtaacagcagccggagatcccacagagatcgtcactgcgggactgatcgaagaagtctacaacgtcaaagcctgtgcatcccagaccccgtgaacagcaaaccgatgatcgtgccactggaaagatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgctattcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtag

SEQ ID NO: 8 - DNA, 1645 bp, synthetic construct
  source 1..1645: mol_type = other DNA
caaagcacttgctactcaatcatggtaaacagcctggtcgcagtccttcacgattcaaactttgccttccgctacgccttccacctgatcatcatagaagacggtgaagtaacagcagccggagatcccacagagatcgtcactgcgggactgatcgaagaagtctacaacgtcaaagcctgtgcatcccagaccccgtgaacagcaaaccgatgatcgtgccactggaaagatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgcttttcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtag

SEQ ID NO: 9 - DNA, 596 bp, synthetic construct
  source 1..596: mol_type = other DNA
cagatggcgcaattaaatcaagatctcagaactcatttttcaatctcttcttttagggcacccgtcatcaattgagctatcggccattcattaaaaactgcgcgtcgatcaaccgaaagctgagtaagcaaaaaattcgcctcttctctatcgtcaagaagcaaagcgcaaccaatcataatctcgctatagttgtctgggtgcattgtttgatcaagattatgcctaaaagatcgaatttctgttctttgtgaatccagtaatccagtttggcgatacaaaatttgccaaccattaagcctgtaaattggcgactcttctcgatctcgttccacgagccaatcattgagagcttgagctgctataagaaatgcagttttcctgctctgctcagaatcagctgccttaattaggcgaagcaccatccatgtggcgagattatcaacatcaacagtttcttcaagcgctgaatagaaatcaacaagcttatccaaatgcagattcaatactgatggaagccatcgattatcaatgagctcgtacggcgtcactcgctcgattgaggagtcgctccgatccgttgaaggtgtcatagc

SEQ ID NO: 10 - DNA, 1959 bp, synthetic construct
  source 1..1959: mol_type = other DNA
tccaaggaagatacacgcccgaagaagctccgttaatggacgctcttctattaagccttttctgatggatacaagcagagaatcagtcatgctaaatatcataatccatcaaacatttgatcggtgtcaatcaatgcgttctggactgaggtgaccaccatgcaagacgaggtggacctagtgatcaccaacccgccgttttctctgttccgtgagttcctgagttggctattacacggtgacgtgttgttttctatcatcggtaacgcgaacgtaatcacatatctaggtgcctgaatctagattaaaactataaacattatttaaaaccattgccttattggagcatgctgcaagcttttcgcggtgggcttgcaacatcttacatcagaatgaatgatgttaaacacctaataagttcaagtagttaaaaggaaatatcaacaatgttacgaaaagcatctatcacgctaatgatatccgttacgctcttaacatgtgcctctccagcacaggcactgtcatcacaagcactctcgtcagaaagctccacttcgcagagtgattctcctacgcaatttgttgcgagtatagctgcgcccttcaataagaacttaacctatgagcagcgcaaagccattcgtttcggacctacgacagaacaagaagcagaacaatgcttggcaaagtatggtcgcgatggcgaggggccatggcctgctattgctatctatcctgactgctccttcgatactatcgaacaagatttctatgatcaagcaatatccaaagccctctcagtaatcagtctgggaagctcctagaagactcgtaacgaccaaaagcaccgagaacagatgcaactcaagcccatttacatctgttctcggtgcaccaaagcacttgctactcaatcatggtaaacagcctggtcgcagtccttcacgattcaaactttgccttccgctacgccttccacctgatcatcatagaagacggtgaagtaacagcagccggagatcccacagagatcgtcactgcgggactgatcgaagaagtctacaacgtcaaagcctgtgcatcccagaccccgtgaacagcaaaccgatgatcgtgccactggaaagatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgacc

SEQ ID NO: 11 - DNA, 1600 bp, synthetic construct
  source 1..1600: mol_type = other DNA
caacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgctattcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtagcagatggcgcaattaaatcaagatctcagaactcatttttcaatctcttcttttagggcacccgtcatcaattgagctatcggccattcattaaaaactgcgcgtcgatcaaccgaaagctgagtaagcaaaaaattcgcctcttctctatcgtcaagaagcaaagcgcaaccaatcataatctcgctatagttgtctgggtgcattgtttgatcaagattatgcctaaaagatcgaatttctgttctttgtgaatccagtaatccagtttggcgatacaaaatttgccaaccattaagcctgtaaattggcgactcttctcgatctcgttccacgagccaatcattgagagcttgagctgctataagaaatgcagttttcctgctctgctcagaatcagctgccttaattaggcgaagcaccatccatgtggcgagattatcaacatcaacagtttcttcaagcgctgaatagaaatcaacaagcttatccaaatgcagattcaatactgatggaagccatcgattatcaatgagctcgtacggcgtcactcgctcgattgaggagtcgctccgatccgttgaaggtgtcatagcaaaatgatgacgcttggaaattacttcatgataagtccactcccctgcaacatcaccagggtgagctatcaacgcaagattccaacgacca

SEQ ID NO: 12 - DNA, 1715 bp, synthetic construct
  source 1..1715: mol_type = other DNA
gcttgcatgcctgcaggtcgactctagaggatcccccaaagcacttgctactcaatcatggtaaacagcctggtcgcagtccttcacgattcaaactttgccttccgctacgccttccacctgatcatcatagaagacggtgaagtaacagcagccggagatcccacagagatcgtcactgcgggactgatcgaagaagtctacaacgtcaaagcctgtgcatcccagaccccgtgaacagcaaaccgatgatcgtgccactggaaagatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgctattcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtaggttttggcggatgagagaagattttcagcctgat

SEQ ID NO: 13 - DNA, 1715 bp, synthetic construct
  source 1..1715: mol_type = other DNA
gcttgcatgcctgcaggtcgactctagaggatcccccaaagcacttgctactcaatcatggtaaacagcctggtcgcagtccttcacgattcaaactttgccttccgctacgccttccacctgatcatcatagaagacggtgaagtaacagcagccggagatcccacagagatcgtcactgcgggactgatcgaagaagtctacaacgtcaaagcctgtgcatcccagaccccgtgaacagcaaaccgatgatcgtgccactggaaagatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgctgtacgagatccgtggcccggtggccgcggaggcggaacgcatggagcttgatgggcataacatcttaaagctcaacacgggaaatccagccgtgttcggattcgatgcccccgacgtgattatgcgtgacatgatcgccaaccttccaacttcccaagggtattccacctccaaaggcattattccggcccggcgagcagtggtcacccgctacgaagttgtgcccggattcccccacttcgatgttgatgatgtgttcttaggcaacggtgtctcagaactaatcaccatgaccacccaagcactcctcaacgacggcgatgaagttcttatccccgcaccggactacccactgtggactgccgcaacctccctggctggtggtaagcctgtgcactacctctgtgatgaggaagatgactggaacccatccatcgaagacatcaagtccaaaatctcagagaaaaccaaagctattgtggtgatcaaccccaacaaccccacgggagctgtctacccgcgccgggtgttggaacaaatcgtcgagattgcacgcgagcatgacctgctgattttggccgatgaaatctacgaccgcattctctacgatgatgccgagcacatcagcctggcaacccttgcaccagatctcctttgcatcacatacaacggtctatccaaggcataccgcgtcgcaggataccgagctggctggatggtattgactggaccaaagcaatacgcacgtggatttattgagggcctcgaactcctcgcaggcactcgactctgcccaaatgtcccagctcagcacgcttttcaggtagctctcggtggacgccagtccatctacgacctcactggcgaacacggccgactcctggaacagcgcaacatggcatggacgaaactcaacgaaatcccaggtgtcagctgtgtgaaaccaatgggagctctatacgcgttccccaagctcgaccccaacgtgtacgaaatccacgacgacacccaactcatgctggatcttctccgtgccgagaaaatcctcatggttcagggcactggcttcaactggccacatcacgatcacttccgagtggtcaccctgccatgggcatcccagttggaaaacgcaattgagcgcctgggtaacttcctgtccacttacaagcagtaggttttggcggatgagagaagattttcagcctgat

SEQ ID NO: 14 - DNA, 1400 bp, synthetic construct
  source 1..1400: mol_type = other DNA
gcagcaagtagcctgggtattcgccacggacgtgaatattgccgaaggcatagtcctcgtgggacttttgctgggcggtaagttgatcgcgtgggtccggggtaatgccataacgaggaacggcaataatcatgcaaccgatctggttttgtgggtcgatctcatgagcaatcttagttgccaaagcacttgctactcaatcatggtaaacagcctggtcgcagtccttcacgattcaaactttgccttccgctacgccttccacctgatcatcatagaagacggtgaagtaacagcagccggagatcccacagagatcgtcactgcgggactgatcgaagaagtctacaacgtcaaagcctgtgcatcccagaccccgtgaacagcaaaccgatgatcgtgccactggaaagatcttaggcagccgtgggattacacccttttagagctagaacagtaaaaattcacccaatagctttcaactacgcacacaaagtggcaacattgagcgggtgactacagacaagcgcaaaacctctaagaccaccgacaccgccaacaaggctgtgggcgcggatcaggcagcgcgtcccactcggcgaacaactcgccgcatcttcgatcagtcggagaagatgaaggacgtgggtaacttcctgtccacttacaagcagtagtagttgttaggattcaccacgaatctcaggatttttgagattcgtggtgaatttttgcgttttccagtcaggctcctgcaactttcggaccgatttcagaggggcggagctggtttgtggtggatccttgaaatggaacctcgcaggaagctttcaggaagaccaagttgggcctaggggtggcgggattgcaaaaatccgtccccggttcgccatgaaatgctgattttgatcgaatctttgcgctaactgtagggcgggttcagggggtgaatgcaccacgagcaacccgaagggtgcgaagtgggcattcgtagaacaatcccagaggaaagccgtacggctttcctcgacatgatcaatcaaggtatgtcaggtcttgctgcgtctacagcggtcggggtcagtgaattcaccgggcgaaagtgggcgaaggccgccggggtgaaactgacccgcggcccgcgaggtggcaatgcttttgacaccgccgagaaacttgagattgcagccagcatgctagagaaaggatgcctaccccgagaaatcggcgagtatgtcggcatgactcgggccaatatatccctatggcgcaaacaaggcccagacaagcttcgccaacgcgcagccaccttgcgcaccggcaagcgagcagctgaattcatccacgccccggtgatgggcccttattatgggccacgcacactccatcaagtgttgcgt

SEQ ID NO: 15 - DNA, 55 bp, synthetic construct
  source 1..55: mol_type = other DNA
cagtgccaagcttgcatgcctgcaggtcgactctagtcacccgctacgaagttgt

SEQ ID NO: 16 - DNA, 33 bp, synthetic construct
  source 1..33: mol_type = other DNA
ctacctgaaaagcgtgctgagctgggacatttg

SEQ ID NO: 17 - DNA, 42 bp, synthetic construct
  source 1..42: mol_type = other DNA
caaatgtcccagctcagcacgcttttcaggtagctctcggtg

SEQ ID NO: 18 - DNA, 57 bp, synthetic construct
  source 1..57: mol_type = other DNA
cagctatgaccatgattacgaattcgagctcggtacccttgttctacgaatgcccac

SEQ ID NO: 19 - DNA, 18 bp, synthetic construct
  source 1..18: mol_type = other DNA
tgtaaaacgacggccagt

SEQ ID NO: 20 - DNA, 18 bp, synthetic construct
  source 1..18: mol_type = other DNA
caggaaacagctatgacc

SEQ ID NO: 21 - DNA, 19 bp, synthetic construct
  source 1..19: mol_type = other DNA
tctatccaaggcataccgc

SEQ ID NO: 22 - DNA, 19 bp, synthetic construct
  source 1..19: mol_type = other DNA
tcccattggtttcacacag

SEQ ID NO: 23 - DNA, 55 bp, synthetic construct
  source 1..55: mol_type = other DNA
cagtgccaagcttgcatgcctgcaggtcgactctagaatgcgttctggactgagg

SEQ ID NO: 24 - DNA, 39 bp, synthetic construct
  source 1..39: mol_type = other DNA
gattgagtagcaagtgctttggtgcaccgagaacagatg

SEQ ID NO: 25 - DNA, 39 bp, synthetic construct
  source 1..39: mol_type = other DNA
catctgttctcggtgcaccaaagcacttgctactcaatc

SEQ ID NO: 26 - DNA, 41 bp, synthetic construct
  source 1..41: mol_type = other DNA
gatttaattgcgccatctgctactgcttgtaagtggacagg

SEQ ID NO: 27 - DNA, 41 bp, synthetic construct
  source 1..41: mol_type = other DNA
cctgtccacttacaagcagtagcagatggcgcaattaaatc

SEQ ID NO: 28 - DNA, 60 bp, synthetic construct
  source 1..60: mol_type = other DNA
cagctatgaccatgattacgaattcgagctcggtacccgctatgacaccttcaacggatc

SEQ ID NO: 29 - DNA, 19 bp, synthetic construct
  source 1..19: mol_type = other DNA
tccaaggaagatacacgcc

SEQ ID NO: 30 - DNA, 19 bp, synthetic construct
  source 1..19: mol_type = other DNA
ggtcgtagatttcatcggc

SEQ ID NO: 31 - DNA, 22 bp, synthetic construct
  source 1..22: mol_type = other DNA
caacggtgtctcagaactaatc

SEQ ID NO: 32 - DNA, 18 bp, synthetic construct
  source 1..18: mol_type = other DNA
tggtcgttggaatcttgc

SEQ ID NO: 33 - DNA, 57 bp, synthetic construct
  source 1..57: mol_type = other DNA
gcttgcatgcctgcaggtcgactctagaggatcccccaaagcacttgctactcaatc

SEQ ID NO: 34 - DNA, 56 bp, synthetic construct
  source 1..56: mol_type = other DNA
atcaggctgaaaatcttctctcatccgccaaaacctactgcttgtaagtggacagg

SEQ ID NO: 35 - DNA, 56 bp, synthetic construct
  source 1..56: mol_type = other DNA
cagtgccaagcttgcatgcctgcaggtcgactctaggcagcaagtagcctgggtat

SEQ ID NO: 36 - DNA, 35 bp, synthetic construct
  source 1..35: mol_type = other DNA
agaagatgaaggacgtgggtaacttcctgtccact

SEQ ID NO: 37 - DNA, 40 bp, synthetic construct
  source 1..40: mol_type = other DNA
agtggacaggaagttacccacgtccttcatcttctccgac

SEQ ID NO: 38 - DNA, 57 bp, synthetic construct
  source 1..57: mol_type = other DNA
cagctatgaccatgattacgaattcgagctcggtacccacgcaacacttgatggagt

SEQ ID NO: 39 - protein, 5 aa, synthetic construct
RRRRR

SEQ ID NO: 40 - protein, 6 aa, synthetic construct
HHHHHH

SEQ ID NO: 41 - protein, 8 aa, synthetic construct
DYKDDDDK

SEQ ID NO: 42 - protein, 8 aa, synthetic construct
WSHPQFEK

SEQ ID NO: 43 - protein, 10 aa, synthetic construct
EQKLISEEDL